SEQUENCE LISTING (30 sequences)

SEQ ID NO: 1 - DNA, 60 bp, Artificial Sequence
  Description of Artificial Sequence Synthetic primer
gactgataatacgactcactatagggcgaatgaagccttccattgccatcattctttgca

SEQ ID NO: 2 - DNA, 44 bp, Artificial Sequence
  Description of Artificial Sequence Synthetic primer
ttagcggccgctcagtatcttttaggcaggccagcaggtcggaa

SEQ ID NO: 3 - DNA, 369 bp, Mus sp.
atgaagccttccattgccatcattctttgcatcttgatcctgggagttgacagccaaagatgggtccagttcatgaaagaagctggtcaagggtctagagacatgtggcgagcctactctgacatgaagaaagctaactggaaaaactcagacaaatacttccatgctcgggggaactatgatgctgcccggaggggtcccgggggagcctgggctgctaaagtcatcagcgatgccagagaggctgttcagaagttcacgggacatggagcagaggactcaagagctgaccagtttgccaatgagtggggccggagtggcaaagaccccaaccacttccgacctgctggcctgcctaaaagatactga

SEQ ID NO: 4 - DNA, 40 bp, Artificial Sequence
  Description of Artificial Sequence Synthetic primer
ttagaattcatgaagccttccattgccatcattctttgca

SEQ ID NO: 5 - DNA, 62 bp, Artificial Sequence
  Description of Artificial Sequence Synthetic primer
gactgataatacgactcactatagggcgatcagtatcttttaggcaggccagcaggtcggaa

SEQ ID NO: 6 - DNA, 369 bp, Mus sp.
tcagtatcttttaggcaggccagcaggtcggaagtggttggggtctttgccactccggccccactcattggcaaactggtcagctcttgagtcctctgctccatgtcccgtgaacttctgaacagcctctctggcatcgctgatgactttagcagcccaggctcccccgggacccctccgggcagcatcatagttcccccgagcatggaagtatttgtctgagtttttccagttagctttcttcatgtcagagtaggctcgccacatgtctctagacccttgaccagcttctttcatgaactggacccatctttggctgtcaactcccaggatcaagatgcaaagaatgatggcaatggaaggcttcat

SEQ ID NO: 7 - DNA, 63 bp, Artificial Sequence
  Description of Artificial Sequence Synthetic primer
gactgataatacgactcactatagggcgaatgtctgatgacaatggatctggtaacaattgca

SEQ ID NO: 8 - DNA, 44 bp, Artificial Sequence
  Description of Artificial Sequence Synthetic primer
ttagcggccgctcaagtgggtgtgctcctggcgatgcaggcata

SEQ ID NO: 9 - DNA, 288 bp, Mus sp.
atgtctgatgacaatggatctggtaacaattgcacaaccaatcatttcctgctctatgggagcctgggactgggagggctcctcctcctgcttgtcatcatcctgttcatctgcctgtgcgggttcagtcagagagtgaagagactggaaaggaatgcccaggtctcagggcaggagccccactatgcatctctccagcagctgccagtgtccagtagtgatatcacagacatgaaagaagacctcagcactgactatgcctgcatcgccaggagcacacccacttga

SEQ ID NO: 10 - DNA, 43 bp, Artificial Sequence
  Description of Artificial Sequence Synthetic primer
ttagaattcatgtctgatgacaatggatctggtaacaattgca

SEQ ID NO: 11 - DNA, 62 bp, Artificial Sequence
  Description of Artificial Sequence Synthetic primer
gactgataatacgactcactatagggcgatcaagtgggtgtgctcctggcgatgcaggcata

SEQ ID NO: 12 - DNA, 288 bp, Mus sp.
tcaagtgggtgtgctcctggcgatgcaggcatagtcagtgctgaggtcttctttcatgtctgtgatatcactactggacactggcagctgctggagagatgcatagtggggctcctgccctgagacctgggcattcctttccagtctcttcactctctgactgaacccgcacaggcagatgaacaggatgatgacaagcaggaggaggagccctcccagtcccaggctcccatagagcaggaaatgattggttgtgcaattgttaccagatccattgtcatcagacat

SEQ ID NO: 13 - DNA, 59 bp, Artificial Sequence
  Description of Artificial Sequence Synthetic primer
gactgataatacgactcactatagggcgaatgtggaaacgatggctggcgctctcgctg

SEQ ID NO: 14 - DNA, 21 bp, Artificial Sequence
  Description of Artificial Sequence Synthetic primer
aggaacagacacagcgattgc

SEQ ID NO: 15 - DNA, 421 bp, Mus sp.
atgtggaaacgatggctggcgctctcgctggtgaccatcgccctggtccacggcgaggaggaacctagaagcaaatccaagatctgcgccaatgtgttttgtggagctggcagggaatgtgccgtcacagagaagggggagcccacgtgcctctgcattgagcaatgcaaacctcacaagaggcctgtgtgtggcagtaatggcaagacctacctcaaccactgtgaacttcatagagatgcctgcctcactggatccaagatccaggttgattatgatgggcactgcaaagaaaagaagtctgcgagtccatctgccagcccagttgtctgctatcaagctaaccgcgatgagctccgacggcgcctcatccagtggctggaagctgagatcattccagatggctggttctctaaaggca

SEQ ID NO: 16 - DNA, 47 bp, Artificial Sequence
  Description of Artificial Sequence Synthetic primer
ggggatatcatgtggaaacgatggctggcgctctcgctggtgaccat

SEQ ID NO: 17 - DNA, 59 bp, Artificial Sequence
  Description of Artificial Sequence Synthetic primer
gactgataatacgactcactatagggcgatgcctttagagaaccagccatctggaatga

SEQ ID NO: 18 - DNA, 421 bp, Mus sp.
tgcctttagagaaccagccatctggaatgatctcagcttccagccactggatgaggcgccgtcggagctcatcgcggttagcttgatagcagacaactgggctggcagatggactcgcagacttcttttctttgcagtgcccatcataatcaacctggatcttggatccagtgaggcaggcatctctatgaagttcacagtggttgaggtaggtcttgccattactgccacacacaggcctcttgtgaggtttgcattgctcaatgcagaggcacgtgggctcccccttctctgtgacggcacattccctgccagctccacaaaacacattggcgcagatcttggatttgcttctaggttcctcctcgccgtggaccagggcgatggtcaccagcgagagcgccagccatcgtttccacat

SEQ ID NO: 19 - DNA, 40 bp, Artificial Sequence
  Description of Artificial Sequence Synthetic primer
aatgcggccgctcacatcgggggcaggcagactttcccac

SEQ ID NO: 20 - DNA, 396 bp, Mus sp.
atgaagtcctgcggccttttacctttcacggtgctccttgctctggggatcctggcaccctggactgtggaaggaggcaaaaatgatgctatcaaaatcggagcctgccctgctaaaaagcctgcccagtgccttaagcttgagaagccacaatgccgtactgactgggagtgcccgggaaagcagaggtgctgccaagatgcttgcggttccaagtgcgtgaatcctgttcccattcgcaaaccagtgtggaggaagcctgggaggtgcgtcaaaactcaggcaagatgtatgatgcttaaccctcccaatgtctgccagagggacgggcagtgtgacggcaaatacaagtgctgtgagggtatatgtgggaaagtctgcctgcccccgatgtga

SEQ ID NO: 21 - DNA, 43 bp, Artificial Sequence
  Description of Artificial Sequence Synthetic primer
attgaattcatgaagtcctgcggccttttacctttcacggtgc

SEQ ID NO: 22 - DNA, 58 bp, Artificial Sequence
  Description of Artificial Sequence Synthetic primer
gactgataatacgactcactatagggcgatcacatcgggggcaggcagactttcccac

SEQ ID NO: 23 - DNA, 396 bp, Mus sp.
tcacatcgggggcaggcagactttcccacatataccctcacagcacttgtatttgccgtcacactgcccgtccctctggcagacattgggagggttaagcatcatacatcttgcctgagttttgacgcacctcccaggcttcctccacactggtttgcgaatgggaacaggattcacgcacttggaaccgcaagcatcttggcagcacctctgctttcccgggcactcccagtcagtacggcattgtggcttctcaagcttaaggcactgggcaggctttttagcagggcaggctccgattttgatagcatcatttttgcctccttccacagtccagggtgccaggatccccagagcaaggagcaccgtgaaaggtaaaaggccgcaggacttcat

SEQ ID NO: 24 - DNA, 49 bp, Artificial Sequence
  Description of Artificial Sequence Synthetic primer
gactgataatacgactcactatagggcgaacaccaacaccagccatgtc

SEQ ID NO: 25 - DNA, 22 bp, Artificial Sequence
  Description of Artificial Sequence Synthetic primer
ctctcagcagtttccccaaatc

SEQ ID NO: 26 - DNA, 313 bp, Mus sp.
acaccaacaccagccatgtcatgcaatatgggaacaaatctatctctaccatgaaagtgatgcagtttcagggaatgaagcacagagccagttcccccatctccctgcctccggtcacacaccttgacctcacccccagccctgacgtgcccctgaccatcttgaagaggaagctgctgagaaccaacgacgtgaaggaatcccagaatctcattgggcagatccagcaatttctggatgccaggcacgtcattgagaagtctgtgcacaagatcgtttccctgctggcgggatttggggaaactgctgagag

SEQ ID NO: 27 - DNA, 20 bp, Artificial Sequence
  Description of Artificial Sequence Synthetic primer
acaccaacaccagccatgtc

SEQ ID NO: 28 - DNA, 51 bp, Artificial Sequence
  Description of Artificial Sequence Synthetic primer
gactgataatacgactcactatagggcgactctcagcagtttccccaaatc

SEQ ID NO: 29 - DNA, 313 bp, Mus sp.
ctctcagcagtttccccaaatcccgccagcagggaaacgatcttgtgcacagacttctcaatgacgtgcctggcatccagaaattgctggatctgcccaatgagattctgggattccttcacgtcgttggttctcagcagcttcctcttcaagatggtcaggggcacgtcagggctgggggtgaggtcaaggtgtgtgaccggaggcagggagatgggggaactggctctgtgcttcattccctgaaactgcatcactttcatggtagagatagatttgttcccatattgcatgacatggctggtgttggtgt

SEQ ID NO: 30 - DNA, 62 bp, Artificial Sequence
  Description of Artificial Sequence Synthetic primer
gactgataatacgactcactatagggcgaatgaagtcctgcggccttttacctttcacggtg

(19) United States
(12) Patent Application Publication
(10) Pub. No.: US 2005/0244849 A1
(43) Pub. Date: Nov. 3, 2005

(54) Screening assays for rheumatoid arthritis

(75) Inventors: Debra D. Pittman, Windham, NH (US); Jeffrey L. Feldman, Arlington, MA (US); Kathleen M. Shields, Harvard, MA (US); William L. Trepicchio, Andover, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Genetics Institute, LLC, Cambridge, MA (US)

(21) Appl. No.: 11/014,625

(22) Filed: Dec. 16, 2004

Related U.S. Application Data
(63) Continuation-in-part of application No. 10/023,451, filed on Dec. 17, 2001.
(60) Provisional application No. 60/255,861, filed on Dec. 15, 2000.

(51) Int. Cl. C12Q 1/68; G01N 33/53; G06F 19/00; G01N 33/48; G01N 33/50

(52) U.S. Cl. 435/6; 702/19; 702/20; 435/7.1

(57) ABSTRACT

The invention provides methods for identifying, designing, and optimizing therapeutics for R.A using as targets one or more genes (and/or their encoded gene products) that have been shown to be up- or down-regulated in cells of R.A. relative to normal counterpart cells. Methods and compositions for diagnostic assays for detecting R.A. are also provided.

### CROSS-REFERENCE TO RELATED APPLICATIONS

This application is a continuation-in-part of U.S. patent application Ser. No. 10/023,451, filed Dec. 17, 2001, which claims the benefit of U.S. Provisional Application No. 60/255,861, filed Dec. 15, 2000, the contents of which applications are herein specifically incorporated by reference in their entireties.

### BACKGROUND OF THE INVENTION

Inflammatory reactions are the cause of a significant number of diseases or disorders, some of which lack appropriate methods of treatment. For example, rheumatoid arthritis (R.A.) is a systematic inflammatory disease that commonly affects the joints, particularly those of the hands and feet. The onset of rheumatoid arthritis can occur slowly, ranging from a few weeks to a few months, or the condition can surface rapidly in an acute manner.

Today, over 2,500,000 individuals are diagnosed with rheumatoid arthritis in the United States alone (1% of population), with some statistics indicating from 6.5 to 8 million potentially afflicted with the disease. Women are affected 2-3 times more often than men. The disease can occur at any age and typically will increase in incidence with age.

The classic early symptoms of rheumatoid arthritis include stiffness, tenderness, fever, subcutaneous nodules, achy joints, and fatigue. The joints of the hands, feet, knees and wrists are most commonly affected, with eventual involvement of the hips, elbows and shoulders. As the joints stiffen and swell, any type of motion becomes very painful and difficult. The more severe cases of rheumatoid arthritis can lead to intense pain and eventual joint destruction. Some 300,000 bone and joint replacement surgical procedures are performed annually in an effort to alleviate the pain and mobility loss resultant from arthritis related joint destruction.

The effective treatment of rheumatoid arthritis has generally comprised a combination of medication, exercise, rest and proper joint protection therapy. The therapy for a particular patient depends on the severity of the disease and the joints that are involved. Aspirin is widely used for pain and to reduce inflammation. In addition to aspirin, non-steroidal anti-inflammatory drugs, corti-costeroids, gold salts, anti-malarials and systemic immunosuppressants are widely used in moderate to advanced cases. The use of steroids and immunosuppressants, however, has significant risks and side effects both in terms of toxicity and vulnerability to potentially lethal conditions.

Thus, there exists a need for methods of diagnosing and treating inflammatory diseases, e.g., rheumatoid arthritis, which do not entail the potentially lethal side effects associated with the treatments described above.

### SUMMARY OF THE INVENTION

This application relates in part to methods for identifying or validating candidate therapeutics for rheumatoid arthritis comprising contacting a compound with a RAGE (AGER) gene or protein, wherein binding of the compound indicates or confirms that it is a candidate therapeutic. The compound may be selected from the following classes: proteins, peptides, peptidomimetics, nucleic acids and small molecules. Binding may be determined using any of a variety of in vitro or in vivo assays, as further described herein.

The present invention also provides methods for identifying a candidate therapeutic for rheumatoid arthritis comprising contacting a compound with a RAGE (AGER) protein, wherein the ability of the compound to inhibit the protein's activity indicates that the compound is a candidate therapeutic. In another embodiment, a method for identifying a candidate therapeutic for rheumatoid arthritis comprises contacting a compound with a RAGE (AGER) gene, wherein the ability to inhibit the gene's activity indicates or confirms that it is a candidate therapeutic. In one embodiment, a method for identifying a candidate therapeutic for rheumatoid arthritis comprises contacting a compound with a RAGE (AGER) gene, wherein the normalization of said gene's expression indicates a candidate therapeutic. Gene expression may be detected using a microarray or by other methods as described herein. In certain embodiments, a method for identifying a candidate therapeutic for treating rheumatoid arthritis comprises comparing the expression profile of a cell incubated with a test compound with at least one expression profile of a counterpart cell derived from a normal subject, wherein a similar expression profile in the two cells indicates that the compound is likely to be effective as a therapeutic for rheumatoid arthritis.

The present invention also provides methods for determining the efficacy of a candidate therapeutic as a drug for rheumatoid arthritis, comprising the steps of: a) contacting a candidate therapeutic to a cell of a subject, and b) determining the ability of the candidate therapeutic to normalize the expression profile for the RAGE (AGER) gene from the cell.

Further features and advantages of the invention will be apparent based on the following Detailed Description and claims.

### DETAILED DESCRIPTION OF THE INVENTION

1. General:

The invention is based at least in part on the discovery of gene expression profiles of cells of subjects having rheumatoid arthritis (R.A.). As described in the Examples, cells from R.A. subjects have genes which are expressed at higher levels (i.e., up-regulated) and genes which are expressed at lower levels (i.e., down-regulated) relative to cells of the same type in subjects which do not have any symptoms of R.A. In particular, as described in the Examples, it has been shown that genes SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6 are expressed at higher levels in the diseased cells relative to the corresponding normal cells. Other genes, e.g., CMAK2B; PLA2G2A; GBAS and SOX15, are down-regulated in the diseased cells relative to the corresponding normal cells. These genes and/or their encoded gene products are novel targets for the development of therapeutics for R.A.

2. Definitions:

As used herein, the following terms and phrases shall have the meanings set forth below. Unless defined otherwise, all technical and scientific terms used herein have the same meaning as commonly understood to one of ordinary skill in the art to which this invention belongs.

“LST-1 (LY117)” refers to a mammalian leukocyte specific transcript 1 gene. SOCS3 is also referred to in the art as D6S49E, LY117, NKp30, activating natural killer receptor p30, B144 protein, and lymphocyte antigen 117, which terms are synonymous with the term LST-1 (LY117) as used herein. In one non-limiting example, a polypeptide sequence encoded by human LST-1 (LY117) may be obtained from GenBank Accession Number AAB86998, and the corresponding mRNA polynucleotide sequence from GenBank Accession Number A F000424. The term LST-1 (LY117) also includes portions of, homologs of, orthologs of, variants of, isoforms of, allelic variants or other sequences at least about 80% identical to any mammalian leukocyte specific transcript 1 gene.

“RAGE (AGER)” refers to a mammalian receptor for advanced glycation end products gene. In one non-limiting example, a polypeptide sequence encoded by human RAGE variant 1 may be obtained from GenBank Accession Number NP_{—}001127, and the corresponding mRNA polynucleotide sequence from GenBank Accession Number NM_{—}001136. In another non-limiting example, a polypeptide sequence encoded by murine RAGE may be obtained from GenBank Accession Number NP_{—}031451, and the corresponding mRNA polynucleotide sequence from GenBank Accession Number NM_{—}007425. The term RAGE also includes portions of, homologs of, orthologs of, variants of, isoforms of, allelic variants or other sequences at least about 80% identical to any mammalian receptor for advanced glycation end products gene.

“SOCS3 (CISH3)” refers to a mammalian Suppressor of Cytokine Signaling 3 gene. SOCS3 is also referred to in the art as cytokine inducible SH2-containing protein 3, STAT-induced STAT inhibitor 3, cytokine-inducible SH2 protein 3, and E2a-Pbx1 target gene in fibroblasts 10, which terms are synonymous with the term SOCS3 (CISH3) as used herein. In one non-limiting example, a polypeptide sequence encoded by human SOCS3 may be obtained from GenBank Accession Number AAD42231, and the corresponding mRNA polynucleotide sequence from GenBank Accession Number AF159854. In another non-limiting example, a polypeptide sequence encoded by murine SOCS3 may be obtained from GenBank Accession Number NP_{—}031733, and the corresponding mRNA polynucleotide sequence from GenBank Accession Number NM_{—}007707. The term SOCS3 also includes portions of, homologs of, orthologs of, variants of, isoforms of, allelic variants or other sequences at least about 80% identical to any mammalian Suppressor of Cytokine Signaling 3 gene.

2. Genes that are Up-Regulated or Down-Regulated in Subjects Having R.A

The invention provides gene expression profiles of R.A. As further described herein in the Examples, the gene expression profiles of the diseased cells of subjects having R.A., indicate that certain genes, e.g., SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6, are significantly up-regulated in these cells relative to their normal counterparts. The expression data also show that certain genes, e.g., CAMK2B, PLA2G2A, GBAS and SOX15, are significantly down-regulated in the diseased cells relative to their normal counterpart cells. These genes and/or their encoded gene products (such as mRNA or proteins) may be used as targets in assays to screen for candidate therapeutics, for example, compounds which modulate the expression of a gene, or compounds which inhibit the activity of a protein, as described in Section 3.0 of this application. Methods for validating and evaluating the efficacy and selectivity of candidate therapeutics for the various targets are described in Sections 4.0 and 5.0 Furthermore, the expression profiles can be used diagnostically and prognostically for R.A, as described in Section 6.0 of this application. Kits comprising reagents and/or compositions for the practice of the methods described in Sections 3.0-6.0 are described in Section 7.0.

3. Assays to Identify or Validate Compounds for Treating R.A.

The expression profiling results described in the Examples indicate that certain genes are expressed at higher levels and certain genes are expressed at lower levels in cells of R.A. patients relative to their expression in normal counterpart cells. Accordingly, reducing the expression of one or more of genes that are up-regulated and/or increasing the expression of one or more genes which are down-regulated in diseased cells may provide a method of treatment of R.A. Furthermore, reducing the activity of a protein that is produced from a gene that is up-regulated and/or stimulating the activity of protein that is produced from a gene that is down-regulating in diseased cells may provide a method of treatment of R.A. Thus, the genes and/or their encoded proteins may serve as targets in methods that evaluate compounds as candidate therapeutics for R.A.

The present invention provides methods for evaluating compounds for their ability to modulate the expression of a target gene or protein by contacting the gene or protein with a compound. In certain embodiments, the compound will be evaluated for its ability to normalize the expression levels (either up-regulate if down-regulated in R.A., or vice versa) of a gene. In certain embodiments, a compound may be evaluated for its ability to down-regulate the activity of a gene whose expression normally promotes R.A. In certain embodiments, a compound may be evaluated for its ability to up-regulate the activity of a gene whose down-regulation normally promotes R.A. In still other embodiments, a compound may be evaluated for its ability to inhibit the activity of a protein whose expression is increased during R.A, or, in the case of a protein whose expression is decreased during R.A., may be evaluated for its ability to stimulate the activity of a protein. In some embodiments, compounds are evaluated for their ability to bind a target gene or protein. These compounds would also have utility in asymptomatic individuals at high risk to develop R.A., as well as in treating those already having R.A.

In embodiments wherein compounds are evaluated for their ability to modulate the activity of or bind a target protein, compounds may be selected from the following classes of compounds: proteins, peptides, peptidomimetics, or small molecules. In other embodiments, wherein compounds are evaluated for their ability to modulate the expression of or bind a target gene, the compounds may be selected from the following classes of compounds: antisense nucleic acids, small molecules, polypeptides, proteins including antibodies, peptidomimetics, or nucleic acid analogs. In some embodiments, the compounds may be selected from a library of compounds. These libraries may be generated using combinatorial synthetic methods.

Those skilled in the art will appreciate from the present description that the ability of a compound to bind or modulate the activity of a gene or protein may be determined by using any of a variety of suitable assays. A person of skill in the art will recognize that in certain screening assays, it will be sufficient to assess the level of expression of a single gene and that in others, the expression of two or more is assessed, whereas still in others, the expression of essentially all the genes involved in R.A. is assessed. Likewise, it will be sufficient to assess the activity of a single protein in some screening assays, whereas in others, the activities of multiple proteins may be assessed. Examples of assays that may be used in the present invention include, but are not limited to, competitive binding assay, direct binding assay, two-hybrid assay, cell proliferation assay, kinase assay, phosphatase assay, nuclear hormone translocator assay, and polymerase chain reaction assay. Further, in certain embodiments of the present invention, the ability of a compound to bind or modulate a target protein or gene may be evaluated by an in vitro assay. In other embodiments, the assay may be an in vivo assay. Such assays are well-known to one of skill in the art and, based on the present description, may be adapted to the methods of the present invention with no more than routine experimentation.

All of the screening methods described in this section may be accomplished by using a variety of assay formats. In light of the present disclosure, those not expressly described herein are nevertheless known and comprehended by one of ordinary skill in the art. The assays may identify agents, e.g., drugs, which are either agonists or antagonists of expression of a target gene of interest, or of a protein:protein or protein-substrate interaction of a target of interest, or of the role of target gene products in the pathogenesis of normal or abnormal cellular physiology, proliferation, and/or differentiation and disorders related thereto. Assay formats which approximate such conditions as formation of protein complexes or protein-nucleic acid complexes, enzymatic activity, and even specific signaling pathways, may be generated in many different forms, as those skilled in the art will appreciate based on the present description and include but are not limited to assays based on cell-free systems, e.g., purified proteins or cell lysates, as well as cell-based assays which utilize intact cells.

In many candidate screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often used as primary screens as they may be generated to permit rapid development and often easy detection of an alteration in a molecular target which is mediated by a test compound. Moreover, the effects of cellular toxicity and/or bioavailability of the test compound may be generally ignored in the in vitro system, the assay instead being focused primarily on the effect of the drug on the molecular target as may be manifest in an alteration of binding affinity with other proteins or changes in enzymatic properties of the molecular target. Accordingly, potential modifiers, e.g., activators or inhibitors of protein-substrate, protein-protein interactions or nucleic acid-protein interactions of interest may be detected in a cell-free assay generated by constitution of function interactions of interest in a cell lysate. In an alternate format, the assay may be derived as a reconstituted protein mixture which, as described below, offers a number of benefits over lysate-based assays.

Below are described several examples of assays which may be used in the methods of the present invention to identify candidate therapeutics.

3. a. Protein Activity and Binding Assays

As those skilled in the art will understand, based on the present description, binding assays may be used to detect agents which, by binding to an active site of a protein, or by disrupting the binding of protein-protein interactions or protein-nucleic acid interactions or the subsequent binding of such a complex or individual protein or nucleic acid to a substrate, may inhibit signaling or other effects resulting from the given interaction. For example, if one polypeptide binds to another polypeptide, drugs may be developed which modulate the activity of the first polypeptide by modulating its binding to the second polypeptide (referred to herein as a “binding partner” or “binding partner”). Cell-free assays may be used to identify compounds which are capable of interacting with a polypeptide or binding partner, to thereby modify the activity of the polypeptide or binding partner. Such a compound may, e.g., modify the structure of the polypeptide or binding partner and thereby effect its activity. Cell-free assays may also be used to identify compounds which bind a polypeptide or modulate the interaction between a polypeptide and a binding partner. In one embodiment, cell-free assays for identifying such compounds consist essentially in a reaction mixture containing a polypeptide and a test compound or a library of test compounds in the presence or absence of a binding partner. A test compound may be, e.g., a derivative of a binding partner, e.g., a biologically inactive peptide, or a small molecule. Agents to be tested for their ability to act as interaction inhibitors may be produced, for example, by bacteria, yeast or other organisms (e.g., natural products), produced chemically (e.g., small molecules, including peptidomimetics), or produced recombinantly. In one embodiment, the candidate therapeutic agent is a small organic molecule.

In one aspect, the present invention provides assays that may be used to screen for agents which modulate protein-protein interactions, nucleic acid-protein interactions, or protein-substrate interactions. For instance, the screening assays of the present invention may be designed to detect agents which disrupt binding of protein-protein interaction binding moieties. In other embodiments, the subject assays will identify inhibitors of the enzymatic activity of a protein or protein-protein interaction complex. In one embodiment, the compound is a mechanism based inhibitor which chemically alters one member of a protein-protein interaction or one chemical group of a protein and which is a specific inhibitor of that member, e.g., has an inhibition constant 10-fold, 100-fold, or 1000-fold different compared to homologous proteins.

In one embodiment of the present invention, assays are provided which detect inhibitory agents on the basis of their ability to interfere with binding of components of a given protein-substrate, protein-protein, or nucleic acid-protein interaction. In an exemplary binding assay, the compound of interest is contacted with a mixture generated from protein-protein interaction component polypeptides. Detection and quantification of expected activity from a given protein-protein interaction provides a means for determining the compound's efficacy at inhibiting (or potentiating) complex formation between the two polypeptides. The efficacy of the compound may be assessed by generating dose response curves from data obtained using various concentrations of the test compound. Moreover, a control assay may also be performed to provide a baseline for comparison. In the control assay, the formation of complexes is quantitated in the absence of the test compound.

Complex formation between component polypeptides, polypeptides and genes, or between a component polypeptide and a substrate may be detected by a variety of techniques, many of which are effectively described above. For instance, modulation in the formation of complexes may be quantitated using, for example, detectably labeled proteins (e.g., radiolabeled, fluorescently labeled, or enzymatically labeled), by immunoassay, or by chromatographic detection.

One aspect of the present invention provides reconstituted protein preparations, e.g., combinations of proteins participating in protein-protein interactions. Such methods are referred to within this section as in vitro.

Accordingly, one exemplary screening assay of the present invention includes the steps of contacting a polypeptide or functional fragment thereof or a binding partner with a test compound or library of test compounds and detecting the formation of complexes. For detection purposes, for example, the molecule may be labeled with a specific marker and the test compound or library oftest compounds labeled with a different marker. Interaction of a test compound with a polypeptide or fragment thereof or binding partner may then be detected by determining the level of the two labels after an incubation step and a washing step. The presence of two labels after the washing step is indicative of an interaction.

An interaction between molecules may also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds may be immobilized on a sensor surface, e.g., which forms one wall of a micro-flow cell. A solution containing the polypeptide, functional fragment thereof, polypeptide analog or binding partner is then flown continuously over the sensor surface. A change in the resonance angle as shown on a signal recording, indicates that an interaction has occurred. This technique is further described, e.g., in BIAtechnology Handbook by Pharmacia.

Another exemplary assay of the present invention includes the steps of (a) forming a reaction mixture including: (i) a polypeptide, (ii) a binding partner, and (iii) a test compound; and (b) detecting interaction of the polypeptide and the binding partner. The polypeptide and binding partner may be produced recombinantly, purified from a source, e.g., plasma, or chemically synthesized, as described herein. A statistically significant change (potentiation or inhibition) in the interaction of the polypeptide and binding partner in the presence of the test compound, relative to the interaction in the absence of the test compound, indicates a potential agonist (mimetic or potentiator) or antagonist (inhibitor) of polypeptide bioactivity for the test compound. The compounds of this assay may be contacted simultaneously. Altematively, a polypeptide may first be contacted with a test compound for an suitable amount of time, following which the binding partner is added to the reaction mixture. The efficacy of the compound may be assessed by generating dose response curves from data obtained using various concentrations of the test compound. Moreover, a control assay may also be performed to provide a baseline for comparison. In the control assay, isolated and purified polypeptide or binding partner is added to a composition containing the binding partner or polypeptide, and the formation of a complex is quantitated in the absence of the test compound.

Complex formation between a polypeptide and a binding partner may be detected by a variety of techniques. Modulation of the formation of complexes may be quantitated using, for example, detectably labeled proteins such as radiolabeled, fluorescently labeled, or enzymatically labeled polypeptides or binding partners, by immunoassay, or by chromatographic detection.

In one embodiment, it will be desirable to immobilize either polypeptide or its binding partner to facilitate separation of complexes from uncomplexed forms of one or both of the proteins, as well as to accommodate automation of the assay. Binding of polypeptide to a binding partner, may be accomplished in any vessel suitable for containing the reactants. Examples include microtitre plates, test tubes, and micro-centrifuge tubes. In one embodiment, a fusion protein may be provided which adds a domain that allows the protein to be bound to a matrix. For example, glutathione-5-transferase/polypeptide (GST/polypeptide) fusion proteins may be adsorbed onto glutathione sepharose beads (Sigma Chemical, St. Louis, Mo.) or glutathione derivatized microtitre plates, which are then combined with the binding partner, e.g., an ³⁵S-labeled binding partner, and the test compound, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes may be dissociated from the matrix, separated by SDS-PAGE (sodium dodecyl sulfate-polyacrylamide gel electrophoresis), and the level of polypeptide or binding partner found in the bead fraction quantitated from the gel using standard electrophoretic techniques such as described in the appended examples.

Other techniques for immobilizing proteins on matrices are also available for use in the subject assays. For instance, either the polypeptide or its cognate binding partner may be immobilized utilizing conjugation of biotin and streptavidin. For instance, biotinylated polypeptide molecules may be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the polypeptide may be derivatized to the wells of the plate, and polypeptide trapped in the wells by antibody conjugation. As above, preparations of a binding partner and a test compound are incubated in the polypeptide presenting wells of the plate, and the amount of complex trapped in the well may be quantitated. Exemplary methods for detecting such complexes, in addition to those described above for the GST-immobilized complexes, include immunodetection of complexes using antibodies reactive with the binding partner, or which are reactive with polypeptide and compete with the binding partner; as well as enzyme-linked assays which rely on detecting an enzymatic activity associated with the binding partner, either intrinsic or extrinsic activity. In an instance of the latter, the enzyme may be chemically conjugated or provided as a fusion protein with the binding partner. To illustrate, the binding partner may be chemically cross-linked or genetically fused with horseradish peroxidase, and the amount of polypeptide trapped in the complex may be assessed with a chromogenic substrate of the enzyme, e.g., 3,3′-diamino-benzadine terahydrochloride or 4-chloro-1-napthol. Likewise, a fusion protein comprising the polypeptide and glutathione-5-transferase may be provided, and complex formation quantitated by detecting the GST activity using 1-chloro-2,4-dinitrobenzene.

For processes that rely on immunodetection for quantitating one of the proteins trapped in the complex, antibodies against the protein, such as anti-polypeptide antibodies, may be used. Alternatively, the protein to be detected in the complex may be “epitope tagged” in the form of a fusion protein which includes, in addition to the polypeptide sequence, a second polypeptide for which antibodies are readily available (e.g., from commercial sources). For instance, the GST fusion proteins described above may also be used for quantification of binding using antibodies against the GST moiety. Other useful epitope tags include myc-epitopes which includes a 10-residue sequence from c-myc, as well as the pFLAG system (International Biotechnologies, Inc., New Haven, Conn.) or the pEZZ-protein A system (Pharmacia, N.J.).

In in vitro embodiments of the present assay, the protein or the set of proteins engaged in a protein-protein, protein-substrate, or protein-nucleic acid interaction comprises a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in a protein-substrate, protein-protein or nucleic acid-protein interaction are present in the mixture to at least 50% purity relative to all other proteins in the mixture, and may be present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure activity resulting from the given protein-substrate, protein-protein interaction, or nucleic acid-protein interaction.

In one embodiment, the use of reconstituted protein mixtures allows more careful control of the protein-substrate, protein-protein, or nucleic acid-protein interaction conditions. Moreover, the system may be derived to favor discovery of inhibitors of particular intermediate states of the protein-protein interaction. For instance, a reconstituted protein assay may be carried out both in the presence and absence of a candidate agent, thereby allowing detection of an inhibitor of a given protein-substrate, protein-protein, or nucleic acid-protein interaction.

Assaying biological activity resulting from a given protein-substrate, protein-protein or nucleic acid-protein interaction, in the presence and absence of a candidate inhibitor, may be accomplished in any vessel suitable for containing the reactants. Examples include microtitre plates, test tubes, and micro-centrifuge tubes.

In one embodiment, it is desirable to immobilize one of the polypeptides to facilitate separation of complexes from uncomplexed forms of one of the proteins, as well as to accommodate automation of the assay. In an illustrative embodiment, a fusion protein may be provided which adds a domain that permits the protein to be bound to an insoluble matrix. For example, protein-protein interaction component fusion proteins may be adsorbed onto glutathione sepharose beads (Sigma Chemical, St. Louis, Mo.) or glutathione derivatized microtitre plates, which are then combined with a potential interacting protein, e.g., an ³⁵S-labeled polypeptide, and the test compound and incubated under conditions conducive to complex formation e.g., at 4° C. in a buffer of 2 mM Tris-HCl (pH 8), 1 nM EDTA, 0.5% Nonidet P-40, and 100 mM NaCl. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes may be dissociated from the matrix, separated by SDS-PAGE, and the level of interacting polypeptide found in the matrix-bound fraction quantitated from the gel using standard electrophoretic techniques.

Another aspect of the present invention provides methods for screening various compounds for their ability to inhibit or reverse the progression of R.A. Such methods are referred to within this section as in vivo as they involve the use of whole cells in culture or the use of animals or samples taken therefrom. In an illustrative embodiment, the subject progenitor cells, and their progeny, can be used to screen various compounds. Such cells can be maintained in minimal culture media for extended periods of time (e.g., for 7-21 days or longer) and can be contacted with any compound, to determine the effect of such compound on one of cellular growth, proliferation or differentiation of progenitor cells in the culture. Detection and quantification of growth, proliferation or differentiation of these cells in response to a given compound provides a means for determining the compound's efficacy at inducing one of the growth, proliferation or differentiation in a given ductal explant. Methods of measuring cell proliferation are well known in the art and most commonly include determining DNA synthesis characteristic of cell replication. However, measurement of protein synthesis may also be used. There are numerous methods in the art for measuring protein synthesis, any of which may be used according to the invention. In an embodiment of the invention, protein synthesis has been determined using a radioactive labeled amino acid (e.g., ³H-leucine) or labeled amino acid or amino acid analogues for detection by immunofluorescence. The efficacy of the compound can be assessed by generating dose response curves from data obtained using various concentrations of the compound. A control assay can also be performed to provide a baseline for comparison. Identification of the progenitor cell population(s) amplified in response to a given test compound can be carried out according to such phenotyping as described above.

In still further embodiments of the present assay, the protein-protein interaction of interest is generated in whole cells, taking advantage of cell culture techniques to support the subject assay. For example, as described below, the protein-protein interaction of interest may be constituted in a eukaryotic cell culture system, including mammalian and yeast cells. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the protein-protein interaction of interest may be endogenous to the cell selected to support the assay. Alternatively, some or all of the components may be derived from exogenous sources. For instance, fusion proteins may be introduced into the cell by recombinant techniques (such as through the use of an expression vector), as well as by microinjecting the fusion protein itself or mRNA encoding the fusion protein.

The cell is ultimately manipulated after incubation with a candidate inhibitor in order to facilitate detection of a protein-protein interaction-mediated signaling event (e.g., modulation of a post-translational medification of a protein-protein interaction component substrate, such as phosphorylation, modulation of transcription of a gene in response to cell signaling, etc.). As described above for assays performed in reconstituted protein mixtures or lysate, the effectiveness of a candidate inhibitor may be assessed by measuring direct characteristics of the protein-protein interaction component polypeptide, such as shifts in molecular weight by electrophoretic means or detection in a binding assay. For these embodiments, the cell will typically be lysed at the end of incubation with the candidate agent, and the lysate manipulated in a detection step in much the same manner as might be the reconstituted protein mixture or lysate, e.g., described above.

Indirect measurement of protein-protein interaction may also be accomplished by detecting a biological activity associated with a protein-protein interaction component that is modulated by a protein-protein interaction mediated signaling event. As set out above, the use of fusion proteins comprising a protein-protein interaction component polypeptide and an enzymatic activity are representative embodiments of the subject assay in which the detection means relies on indirect measurement of a protein-protein interaction component polypeptide by quantitating an associated enzymatic activity.

In other embodiments, the biological activity of a nucleic acid-protein, protein-substrate or protein-protein interaction component polypeptide may be assessed by monitoring changes in the phenotype of the targeted cell. For example, the detection means may include a reporter gene construct which includes a transcriptional regulatory element that is dependent in some form on the level of an interaction component or a interaction component substrate. The protein interaction component may be provided as a fusion protein with a domain which binds to a DNA element of the reporter gene construct. The added domain of the fusion protein may be one which, through its DNA-binding ability, increases or decreases transcription of the reporter gene. Whichever the case may be, its presence in the fusion protein renders it responsive to the protein-protein interaction-mediated signaling pathway. Accordingly, the level of expression of the reporter gene will vary with the level of expression of the protein interaction component.

In yet another embodiment, the protein-protein interaction component or potential interacting polypeptide may be used to generate an two-hybrid or interaction trap assay for subsequently detecting agents which disrupt binding of the interaction components to one another.

In a particular embodiment, the method comprises the use of chimeric genes which express hybrid proteins. To illustrate, a first hybrid gene comprises the coding sequence for a DNA-binding domain of a transcriptional activator may be fused in frame to the coding sequence for a “bait” protein, e.g., a protein-protein interaction component polypeptide of sufficient length to bind to a potential interacting protein. The second hybrid protein encodes a transcriptional activation domain fused in frame to a gene encoding a “fish” protein, e.g., a potential interacting protein of sufficient length to interact with the protein-protein interaction component polypeptide portion of the bait fusion protein. If the bait and fish proteins are able to interact, e.g., form a protein-protein interaction component complex, they bring into close proximity the two domains of the transcriptional activator. This proximity causes transcription of a reporter gene which is operably linked to a transcriptional regulatory site responsive to the transcriptional activator, and expression of the reporter gene may be detected and used to score for the interaction of the bait and fish proteins.

In accordance with the present invention, the method includes providing a host cell, such as a yeast cell. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as a protein-protein interaction component polypeptide sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, λcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction. In certain embodiments, any enzymatic activity associated with the bait or fish proteins is inactivated, e.g., dominant negative or other mutants of a protein-protein interaction component may be used.

Continuing with the illustrated example, the protein-protein interaction component-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of a protein-protein interaction component/interacting protein complex results in a detectable signal produced by the expression of the reporter gene. Accordingly, the level of formation of a complex in the presence of a test compound and in the absence of the test compound may be evaluated by detecting the level of expression of the reporter gene in each case.

Various reporter constructs may be used in accord with the methods of the invention and include, for example, reporter genes which produce such detectable signals as selected from the group consisting of an enzymatic signal, a fluorescent signal, a phosphorescent signal and drug resistance. The reporter gene product may be a detectable label, such as luciferase, β-lactamase or β-galactosidase, and is produced in the intact cell. The label may be measured in a subsequent lysate of the cell. However, the lysis step may be avoided, and providing a step of lysing the cell to measure the label will typically only be employed where detection of the label cannot be accomplished in whole cells.

Moreover, in the whole cell embodiments of the subject assay, the reporter gene construct may provide, upon expression, a selectable marker. A reporter gene includes any gene that expresses a detectable gene product, which may be RNA or protein. Reporter genes include those that are readily detectable. The reporter gene may also be included in the construct in the form of a fusion gene with a gene that includes desired transcriptional regulatory sequences or exhibits other desirable properties. For instance, the product of the reporter gene may be an enzyme which confers resistance to antibiotic or other drug, or an enzyme which complements a deficiency in the host cell (e.g., thymidine kinase or dihydrofolate reductase). To illustrate, the aminoglycoside phosphotransferase encoded by the bacterial transposon gene Tn5 neo may be placed under transcriptional control of a promoter element responsive to the level of a protein-protein interaction component polypeptide present in the cell. Such embodiments of the subject assay are particularly amenable to high throughput analysis in that proliferation of the cell may provide a simple measure of inhibition of an interaction.

Reporter genes further include, but are not limited to CAT (chloramphenicol acetyl transferase) luciferase, and other enzyme detection systems, such as P-galactosidase, β-lactamase, human placental secreted alkaline phosphatase.

The amount of transcription from the reporter gene may be measured using any method known to those of skill in the art to be suitable. For example, specific mRNA expression may be detected using Northern blots or specific protein product may be identified by a characteristic stain, western blots or an intrinsic activity.

In certain embodiments, the product of the reporter gene is detected by an intrinsic activity associated with that product. For instance, the reporter gene may encode a gene product that, by enzymatic activity, gives rise to a detection signal based on color, fluorescence, or luminescence.

The amount of expression from the reporter gene is then compared to the amount of expression in either the same cell in the absence of the test compound or it may be compared with the amount of transcription in a substantially identical cell that lacks a component of the protein-protein interaction of interest.

3. b. Gene Expression Assays

In still other embodiments, assays may be conducted to identify compounds that modulate the activity (e.g. expression) of a gene. In one embodiment, the ability of a compound to up- or down-regulate the level of expression of a gene is determined by phenotypic analysis of the cell, in particular by determining whether the cell adopts a phenotype that is more reminiscent of that of a normal cell than that of a cell characteristic of R.A. In one embodiment, the level of expression of a gene is modulated, and the level of expression of at least one gene characteristic of R.A. is determined, e.g., by using a microarray having probes to the one or more genes. If the normalization of expression of the gene results in at least some normalization of the gene expression profile in the diseased cell, then normalizing the expression of the gene in a subject having R.A. is expected to improve R.A. If, however, the normalization of expression of the gene does not result in at least some normalization of the gene expression profile in the diseased cell, normalizing the expression of the gene in a subject having R.A. is not expected to improve R.A. In certain embodiments, the expression level of two or more genes which are up- or down-regulated in R.A. is modulated and the effect on the diseased cell is determined.

In one embodiment, an agent which modulates the expression of a gene of interest is identified by contacting cells expressing the gene with test compounds, and monitoring the level of expression of the gene. Alternatively, compounds which modulate the expression of gene X can be identified by conducting assays using the promoter region of a gene and screening for compounds which modify binding of proteins to the promoter region. The nucleotide sequence of the promoter may be described in a publication or available in GenBank. Alternatively, the promoter region of the gene can be isolated, e.g., by screening a genomic library with a probe corresponding to the gene. Such methods are known in the art.

One cell for use in these assays is a cell characteristic of R.A. that can be obtained from a subject and, e.g., established as a primary cell culture. The cell can be immortalized by methods known in the art, e.g., by expression of an oncogene or large T antigen of SV40. Alternatively, cell lines corresponding to such a diseased cell can be used. Examples include RAW cells and THP1 cells. However, prior to using such cell lines, it may be to confirm that the gene expression profile of the cell line corresponds essentially to that of a cell characteristic of R.A. This can be done as described in details herein.

Modulating the expression of a gene in a cell can be achieved, e.g., by contacting the cell with an agent that increases the level of expression of the gene or the activity of the polypeptide encoded by the gene. Increasing the level of a polypeptide in a cell can also be achieved by transfecting the cell, transiently or stably, with a nucleic acid encoding the polypeptide. Decreasing the expression of a gene in a cell can be achieved by inhibiting transcription or translation of the gene or RNA, e.g., by introducing antisense nucleic acids, ribozymes or siRNAs into the cells, or by inhibiting the activity of the polypeptide encoded by the gene, e.g., by using antibodies or dominant negative mutants. These methods are further described below in the context of therapeutic methods.

A nucleic acid encoding a particular polypeptide can be obtained, e.g., by RT-PCR from a cell that is known to express the gene. Primers for the RT-PCR can be derived from the nucleotide sequence of the gene encoding the polypeptide. Amplified DNA can then be inserted into an expression vector, according to methods known in the art and transfected into diseased cells of R.A. In a control experiment, normal counterpart cells can also be transfected. The level of expression of the polypeptide in the transfected cells can be determined, e.g., by electrophoresis and staining of the gel or by Western blot using an a agent that binds the polypeptide, e.g., an antibody. The level of expression of one or more genes which are up- or down-regulated in R.A. can then be determined in the transfected cells having elevated levels of the polypeptide. In one embodiment, the level of expression is determined by using a microarray. For example, RNA is extracted from the transfected cells, and used as target DNA for hybridization to a microarray, as further described herein.

Although microarrays may be used in these embodiments, various other methods of detection of gene expression are available. The following paragraphs describe a few exemplary methods for detecting and quantifying gene expression. Where the first step of the methods includes isolation of mRNA from cells, this step may be conducted as described above. Labeling of one or more nucleic acids may be performed as described above.

In one embodiment, mRNA obtained form a sample is reverse transcribed into a first cDNA strand and subjected to PCR, e.g., RT-PCR. House keeping genes, or other genes whose expression does not vary can be used as internal controls and controls across experiments. Following the PCR reaction, the amplified products can be separated by electrophoresis and detected. By using quantitative PCR, the level of amplified product will correlate with the level of RNA that was present in the sample. The amplified samples can also be separated on a agarose or polyacrylamide gel, transferred onto a filter, and the filter hybridized with a probe specific for the gene of interest. Numerous samples can be analyzed simultaneously by conducting parallel PCR amplification, e.g., by multiplex PCR.

A quantitative PCR technique that can be used is based on the use of TaqMan probes. Specific sequence detection occurs by amplification of target sequences in the PE Applied Biosystems 7700 Sequence Detection System in the presence of an oligonucleotide probe labeled at the 5′ and 3′ ends with a reporter and quencher fluorescent dye, respectively (FQ probe), which anneals between the two PCR primers. Only specific product will be detected when the probe is bound between the primers. As PCR amplification proceeds, the 5′-nuclease activity of Taq polymerase initially cleaves the reporter dye from the probe. The signal generated when the reporter dye is physically separated from the quencher dye is detected by measuring the signal with an attached CCD camera. Each signal generated equals one probe cleaved which corresponds to amplification of one target strand. PCR reactions may be set up using the PE Applied Biosystem TaqMan PCR Core Reagent Kit according to the instructions supplied.

In another embodiment, mRNA levels is determined by dotblot analysis and related methods. In one embodiment, a specified amount of RNA extracted from cells is blotted (i.e., non-covalently bound) onto a filter, and the filter is hybridized with a probe of the gene of interest. Numerous RNA samples can be analyzed simultaneously, since a blot can comprise multiple spots of RNA. Hybridization is detected using a method that depends on the type of label of the probe. In another dotblot method, one or more probes of one or more genes which are up- or down-regulated in R.A. are attached to a membrane, and the membrane is incubated with labeled nucleic acids obtained from and optionally derived from RNA of a cell or tissue of a subject. Such a dotblot is essentially an array comprising fewer probes than a microarray.

3. c. Exemplary Compounds that may be Candidate Therapeutics

(i) Antisense Nucleic Acids

One method for decreasing the level of expression of a gene is to introduce into the cell antisense molecules which are complementary to at least a portion of the gene or RNA of the gene. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Antisense nucleic acids may be of at least six nucleotides and may be oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane, hybridization-triggered cleavage agents or intercalating agents. In one aspect of the invention, an antisense oligonucleotide is provided, e.g., as single-stranded DNA. The oligonucleotide may be modified at any position on its structure with constituents generally known in the art. For example, the antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-α-anomeric oligonucleotide. An α-anomeric oligonucleotide forms specific double-stranded hybrids with complementary RNA in which, contrary to the usual β-units, the strands run parallel to each other.

The oligonucleotide may be conjugated to another molecule, e.g., a peptide, hybridization triggered cross-linking agent transport agent, hybridization-triggered cleavage agent, etc. An antisense molecule can be a “peptide nucleic acid” (PNA). The terminal lysine confers solubility to the composition. PNAs bind complementary single stranded DNA or RNA and stop transcript elongation, and may be pegylated to extend their lifespan in the cell.

The antisense nucleic acids of the invention comprise a sequence complementary to at least a portion of a target RNA species. The synthesized antisense oligonucleotides can then be administered to a cell in a controlled manner. For example, the antisense oligonucleotides can be placed in the growth environment of the cell at controlled levels where they may be taken up by the cell. The uptake of the antisense oligonucleotides can be assisted by use of methods well known in the art.

In an alternative embodiment, the antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Promoters may be controllable or inducible by the administration of an exogenous moiety in order to achieve controlled expression of the antisense oligonucleotide. Such controllable promoters include the Tet promoter. Other usable promoters for mammalian cells include, but are not limited to: the SV40 early promoter region, the promoter contained in the 3′ long terminal repeat of Rous sarcoma virus, the herpes thymidine kinase promoter. the regulatory sequences of the metallothionein gene.

(ii) Ribozymes

In another embodiment, the level of a particular mRNA or polypeptide in a cell is reduced by introduction of a ribozyme into the cell or nucleic acid encoding such. Ribozyme molecules designed to catalytically cleave mRNA transcripts can also be introduced into, or expressed, in cells to inhibit expression of the gene. One commonly used ribozyme motif is the hammerhead, for which the substrate sequence requirements are minimal.

(iii) siRNAs

Another method for decreasing or blocking gene expression is by introducing double stranded small interfering RNAs (siRNAs), which mediate sequence specific mRNA degradation. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells.

(iv) Triplex Formation

Gene expression can be reduced by targeting deoxyribonucleotide sequences complementary to the regulatory region of the target gene (i.e., the gene promoter and/or enhancers) to form triple helical structures that prevent transcription of the gene in target cells in the body.

(v) Aptamers

In a further embodiment, RNA aptamers can be introduced into or expressed in a cell. RNA aptamers are specific RNA ligands for proteins, such as for Tat and Rev that can specifically inhibit their translation.

(vi) Dominant negative mutants

Another method of decreasing the biological activity of a polypeptide is by introducing into the cell a dominant negative mutant. A dominant negative mutant polypeptide will interact with a molecule with which the polypeptide normally interacts, thereby competing for the molecule, but since it is biologically inactive, it will inhibit the biological activity of the polypeptide. A dominant negative mutant can be created by mutating the substrate-binding domain, the catalytic domain, or a cellular localization domain of the polypeptide. Preferably, the mutant polypeptide will be overproduced. Point mutations are made that have such an effect. In addition, fusion of different polypeptides of various lengths to the terminus of a protein can yield dominant negative mutants.

(vi) Agents, e.g. Small Molecules or Polypeptides, Inhibiting Transcription or Polypeptide Activity

In another embodiment, a compound decreasing the expression of the target gene or the activity of its encoded polypeptide is administered to a subject having R.A., such that the level of the polypeptide in the diseased cells decreases, and the disease is improved. Compounds may be known in the art or can be identified as described above.

Inhibitors of the polypeptide can also be agents which bind to the polypeptide, and thereby prevent it from functioning normally, or which degrades or causes the polypeptide to be degraded. For example, such an agent can be an antibody or derivative thereof which interacts specifically with the polypeptide. Example antibodies are monoclonal antibodies, humanized antibodies, human antibodies, and single chain antibodies. Such antibodies can be prepared and tested as known in the art or can be identified as further described herein.

4. Validation of Targets

4.a. In Vitro Validation

Targets may be validated by any of a number of cell-based assays, representative of different mechanisms of disease pathology, and also by additional validation experiments. For example, the gene and pathway mining approach leverages a large number of novel genes and proteins with significant homology to inflammatory cytokines, chemokines and their receptors, cellular activation antigens and pharmaceutically tractable intracellular proteins that regulate inflammatory processes. Targets may be further characterized at the expression level, using the yeast two-hybrid platform to map relevant pathways. Various combinations of additional detailed ad hoc biochemical, immunohistochemical, cellular and in vivo animal validation studies, as well as the determination of SNPs and their relevance and bioinformatics analyses provide a high degree of validation for our targets.

For example, the efficacy of the candidate therapeutics may be tested by administering a candidate therapeutic to a test animal and monitoring inhibition of the progress of rheumatoid arthritis or at least one symptom thereof. In another example, expression of one or more genes characteristic of rheumatoid arthritis may also be measured before and after administration of a candidate therapeutic to an animal or cell. A normalization of the expression of one or more of these genes is indicative of the efficiency of the compound for treating rheumatoid arthritis.

Exemplary cell lines and cell cultures for validation of rheumatoid arthritis therapeutics (which also may be used in whole cell in vitro candidate therapeutic screening) include cells and cell lines derived from the synovial tissues of subjects having R.A., cells and cell lines derived from subjects having rheumatoid arthritis-associated HLA-DR alleles, cells and cell lines derived from animal models of R.A., cells transfected with a R.A.-associated gene (such as a target of the invention, and T cells and cell lines derived from subjects having R.A.

Cell lines and cell cultures may be cultured using well-known techniques of cell culture. Suitable media for culture include natural media based on tissue extracts and bodily fluids as well chemically defined media. Media suitable for use with the present invention include media containing serum as well as media that is serum-free. Serum may be from any source, including calf, fetal bovine, horse, and human serum. Any selected medium may contain one or more of the following in any suitable combination: basal media, water, buffers, free-radical scavengers, detergents, surfactants, polymers, cellulose, salts, amino acids, vitamins, carbon sources, organic supplements, hormones, growth factors, antibiotics, nutrients and metabolites, lipids, minerals, and inhibitors. Media may be selected or developed so that a particular pH, CO₂ tension, oxygen tension, osmolality, viscosity, and/or surface tension results from the composition of the medium. The incubation steps of the above method may be accomplished by maintaining the cell cultures in an environment wherein temperature and atmosphere are controlled. The culture conditions may be a ltered to maintain cellular proliferation and contractile activity in the c ell cultures (optimum culture conditions are described below).

Cells, tissues, or other samples taken from animal models (described below) may be used in the methods. Tissues and samples may be extracted from the animals using a variety of methods known in the art, for example, surgical resection, withdrawal of blood or other bodily fluid, urine collection, swabbing, and the like. Examples of experiments that can be performed to evaluate the cells and/or tissues and or samples from the animals include, but are not limited to, morphological examination of cells; histological examination of synovial tissue, of joint tissue; evaluation of DNA replication and/or expression; assays to evaluate enzyme activity; and assays studying programmed cell death, or apoptosis. The methods to perform such experiments are standard and are well known in the art.

4.b. In Vivo Validation

Validation of specific targets or therapeutics may be performed in appropriate animal models for RA, and transgenic mouse models. Such models are also useful for testing and screening of compounds for the treatment of chronic and acute inflammatory disease to identify candidate therapeutics. Drug screening protocols will generally include a panel of animals, for example a test compound or combination of test compounds, and negative and/or positive controls, where the positive controls may be known immunosuppressive agents. Such panels may be treated in parallel, or the results of a screening assay may be compared to a reference database. Suitable animal models and their use in developing therapeutics for rheumatoid arthritis are known in the art. A wide variety of assays may be used for these purposes, including histological analysis of effectiveness, determination of the localization of drugs after administration, labeled in vitro protein-protein binding assays, protein-DNA binding assays, electrophoretic mobility shift assays, immunoassays for protein binding, and the like. Depending on the particular assay, whole animals may be used, or cells derived therefrom, particularly skin cells, e.g. keratinocytes. Cells may be freshly isolated from an animal, or may be immortalized in culture.

For screening assays that use whole animals, a candidate agent or treatment is applied to the subject animals. Typically, a group of animals is used as a negative, untreated or placebo-treated control, and a test group is treated with the candidate therapy. Generally a plurality of assays are run in parallel with different agent dose levels to obtain a differential response to the various dosages. The dosages and routes of administration are determined by the specific compound or treatment to be tested, and will depend on the specific formulation, stability of the candidate agent, response of the animal, etc.

The analysis may be directed towards determining effectiveness in prevention of disease induction, where the treatment is administered before induction of the disease, i.e. prior to injection of the T cells and/or pro-inflammatory cytokine. Alternatively, the analysis is directed toward regression of existing lesions, and the treatment is administered after initial onset of the disease, or establishment of moderate to severe disease. Frequently, treatment effective for prevention is also effective in regressing the disease.

In either case, after a period of time sufficient for the development or regression of the disease, the animals are assessed for impact of the treatment, by visual, histological, immunohistological, and other assays suitable for determining effectiveness of the treatment. The results may be expressed on a semi-quantitative or quantitative scale in order to provide a basis for statistical analysis of the results.

5. Efficacy and Selectivity Studies

The efficacy of the compounds may then be tested in additional in vitro assays and in vivo. A test compound may be administered to a cell or tissue produced by the methods of the invention and at least one characteristic or behavior of the tissue or cell monitored. For example, expression of one or more target genes characteristic of a particular disorder, proliferative state, or differentiation state may also be measured before and after administration of the test compound to the tissue or cell. A normalization of the expression of one or more of these target genes is indicative of the efficiency of the compound for treating disorders in the animal. In another example, the activity of a target protein may be monitored before and after administration of the test compound to a tissue or cell.

The efficacy of the compound can be assessed by generating dose response curves from data obtained using various concentrations of the compound. A control assay can also be performed to provide a baseline for comparison. The data obtained from the cell culture assays and animal studies may be used in formulating a range of dosage for use in humans. The dosage of any supplement, or alternatively of any components therein, lies generally within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

In another embodiment of the invention, a drug is developed by rational drug design, i.e., it is designed or identified based on information stored in computer readable form and analyzed by algorithms. More and more databases of expression profiles are currently being established, numerous ones being publicly available. The present invention provides expression profiles as well as methods for generating them (see next section). By screening such databases for the description of drugs affecting the expression of at least some of the genes characteristic of a disorder in a manner similar to the change in gene expression profile from a diseased cell to that of a normal cell corresponding to the diseased cell, compounds may be identified which normalize gene expression in a diseased cell. Derivatives and analogues of such compounds may then be synthesized to optimize the activity of the compound, and tested and optimized as described above.

The selectivity of a candidate therapeutic can be further evaluated by comparing its activity on a target to its activity on other genes or proteins. For example, the selectivity of a candidate therapeutic with respect to a target gene or protein may be expressed by comparison to another compound, using the respective values of Kd (i.e., the dissociation constants for each modulator-druggable region complex) or, in cases where a biological effect is observed below the Kd, the ratio of the respective EC50's (i.e., the concentrations that produce 50% of the maximum response for the modulator interacting with each druggable region).

Once compounds have been identified that show activity as inhibitors of target function, a program of optimization can be undertaken in an effort to improve the potency and or selectivity of the activity. This analysis of structure-activity relationships (SAR) typically involves of iterative series of selective modifications of compound structures and their correlation to biochemical or biological activity. Families of related compounds can be designed that all exhibit the desired activity, with certain members of the family, namely those possessing suitable pharmacological profiles, potentially qualifying as therapeutic candidates. In addition to designing and/or identifying a chemical entity to associate with a target, as described above, the same techniques and methods may be used to design and/or identify chemical entities that either associate, or do not associate, with affinity regions, selectivity regions or undesired regions of protein or gene targets. By such methods, selectivity for one or a few targets, or alternatively for multiple targets, from the same species or from multiple species, can be achieved.

For example, a compound may be designed and/or identified for which the binding energy for one druggable region, e.g., an affinity region or selectivity region, is more favorable than that for another region, e.g., an undesired region, by about 20%, 30%, 50% to about 60% or more. It may be the case that the difference is observed between (a) more than two regions, (b) between different regions (selectivity, affinity or undesirable) from the same target, (c) between regions of different targets, (d) between regions of homologs from different species, or (e) between other combinations. Alternatively, the comparison may be made by reference to the Kd, usually the apparent Kd, of said chemical entity with the two or more regions in question.

In another aspect, prospective compounds are screened for binding to two nearby druggable regions on a target protein or gene. For example, a compound that binds a first region of a target polypeptide does not bind a second nearby region. Binding to the second region can be determined by monitoring changes in a different set of amide chemical shifts in either the original screen or a second screen conducted in the presence of a candidate therapeutic (or potential modulator) for the first region. From an analysis of the chemical shift changes, the approximate location of a potential modulator for the second region is identified. Optimization of the second modulator for binding to the region is then carried out by screening structurally related compounds (e.g., analogs as described above). When modulators for the first region and the second region are identified, their location and orientation in the ternary complex can be determined experimentally. On the basis of this structural information, a linked compound, e.g., a consolidated modulator, is synthesized in which the modulator for the first region and the modulator for the second region are linked. In certain embodiments, the two modulators are covalently linked to form a consolidated modulator. This consolidated modulator may be tested to determine if it has a higher binding affinity for the target than either of the two individual modulators. A consolidated modulator is selected as a modulator when it has a higher binding affinity for the target than either of the two modulators. Larger consolidated modulators can be constructed in an analogous manner, e.g., linking three modulators which bind to three nearby regions on the target to form a multilinked consolidated modulator that has an even higher affinity for the target than the linked modulator. In this example, it is assumed that is desirable to have the modulator bind to all the druggable regions. However, it may be the case that binding to certain of the druggable regions is not desirable, so that the same techniques may be used to identify modulators and consolidated modulators that show increased specificity based on binding to at least one but not all druggable regions of a target.

6. Exemplary Diagnostic Tools and Assays

Diagnostic methods of the invention involve measuring the level of expression of one or more genes that are up- or down-regulated in R.A. in a cell of a patient, and comparing these levels of expression to the level of expression of the genes in other samples, which levels of expression may be present in a computer readable medium and analyzed with a computer.

(i) In one embodiment, the invention provides a method for determining whether a subject has or is likely to develop R.A., comprising determining the level of expression of one or more genes which are up- or down-regulated in R.A. in a cell of the subject and comparing these levels of expression with the levels of expression of the genes in a diseased cell of a subject known to have R.A. A similar level of expression of the genes in the two cells is indicative that the subject has or is likely to develop R.A. or at least a symptom thereof. In one embodiment, the cell of the subject is essentially of the same type as that which is diseased in R.A.

(ii) In another embodiment the expression profile data of the invention can be used to confirm that a subject has R.A., and in particular, that the subject does not have a disease that is merely related R.A. This can be important, in particular, in designing an optimal therapeutic regimen for the subject. It has been described in the art that expression profiles can be used to distinguish one type of disease from a similar disease. Accordingly, the expression profiles of the invention allow the distinction of R.A. from related diseases. In one embodiment, the level of expression of one or more genes which are up- or down-regulated in R.A. is determined in a cell of the subject, for example a cell which corresponds to a diseased cell in R.A. A level of expression of one or more genes that is more similar to that in a cell characteristic of R.A. than to that of cells of related diseases indicates that the subject has R.A., rather than a disease related to R.A.

Prior to using this method for determining whether the subject has R.A. or a related disease, it may be necessary to first determine the expression profile of cells of diseases that are similar to R.A. This can be undertaken using the same microarray as the one that was used to identify the genes characteristic of R.A., and according to methods further described herein.

(iii) In yet another embodiment, the invention provides methods for determining the stage of R.A. in the subject. In one embodiment, the level of expression of one or more genes that are up- or down-regulated in R.A., in particular, whose level of expression varies with the stage of the disease is determined in a cell of a subject. A level of expression of one or more genes that is more similar to that of one stage of the disease (stage “a”) relative to that in other stages of the disease indicates that the disease of the subject is in stage a.

This assay may require the preliminary determination of expression profiles in different stages of R.A. Such expression data can be obtained by, e.g., using microarrays with target nucleic acids made from RNA of patients at different stages of the disease.

(iv) The method can also be used to determine the efficacy of a therapy in a subject. Accordingly, in one embodiment, the level of expression of one or more genes which are up- or down-regulated in R.A. is determined in a subject before the treatment and one or more times during the treatment. For example, a sample of RNA can be obtained from the subject before the beginning of the therapy and every 12, 24 or 72 hours during the therapy. Samples can also be analyzed once a week or once a month. Changes in expression levels of the genes over time and relative to diseased cells and normal cells will indicate whether the therapy is effective. For example, expression levels that are more similar to those in normal cells or in less advanced stages of the disease relative to the stage the subject was in, indicates that the therapy is effective.

(v) In yet another embodiment, the invention provides a method for determining the likelihood of success of a particular therapy in a subject having R.A. In one embodiment, a subject is started on a particular therapy, and the effectiveness of the therapy is determined, e.g., by determining the level of expression of one or more genes characteristic of R.A. in a cell of the subject. A normalization of the level of expression of these genes, i.e., a change in the expression level of the genes such that their level of expression resembles more that of a non diseased cell, indicates that the treatment should be effective in the subject. On the other hand, the absence of normalization of the level of expression of the genes characteristic of R.A. indicates that the treatment is not likely to be effective in the subject. This method may be able to predict that a treatment is effective before any alleviation of symptoms becomes apparent.

Prediction of the outcome of a treatment of R.A. in a subject can also be undertaken in vitro. In one embodiment, cells are obtained from a subject to be evaluated for responsiveness to the treatment, and incubated in vitro with the therapeutic drug or metabolized form thereof. The level of expression of one or more genes which are up- or down-regulated in R.A. is measured in the cells and these values are compared to the level of expression of these one or more genes in a cell which is a normal counterpart cell of a cell characteristic of R.A. The level of expression can also be compared to that in other diseased cells. A level of expression of one or more genes in the cells of the subject after incubation with the drug that is similar to their level of expression in a normal cell and different from that in a diseased cell is indicative that it is likely that the subject will respond positively to a treatment with the drug. On the contrary, levels of expressions that are more similar to levels of expression in a diseased cell than that in a normal cell is indicative that it is likely that the subject will not respond positively to a treatment with the drug.

Since it is possible that a drug for treating R.A. does not act directly on the diseased cells, but is, e.g., metabolized, or acts on another cell which then secretes a factor that will effect the diseased cells, the above assay can also be conducted in a tissue sample of a subject, which contains cells other than the diseased cells. For example, a tissue sample comprising diseased cells is obtained from a subject; the tissue sample is incubated with the potential drug; optionally one or more diseased cells are isolated from the tissue sample, e.g., by microdissection or Laser Capture Microdissection; and the expression level of one or more genes characteristic of R.A. is examined.

(vi) The invention also provides methods for selecting a particular therapy for an R.A. patient from a selection of several different therapies. Certain subjects having R.A. may respond better to one type of therapy than to another type of therapy. In one embodiment, the method comprises comparing the expression level of at least one gene that is up- or down-regulated in R.A. in the patient with that in cells of R.A. subjects that were treated in vitro or in vivo with one of several therapeutic drugs, which subjects are responders or non responders to one of the therapeutic drugs, and identifying the cell which has the most similar level of expression of that in the patient, to thereby identify a therapy for the patient. The method may further comprise administering the therapy to the subject.

A person of skill in the art will recognize that in certain diagnostic and prognostic assays, it will be sufficient to assess the level of expression of a single gene that is up- or down-regulated in R.A., and that in others, the expression of a plurality, e.g., two or more genes, is assessed.

A person of skill in the art will also recognize that expression levels can be measured in a single cell or in a plurality of cells, e.g., two or more cells. In one embodiment, the method comprises determined expression levels in a cell or tissue sample, e.g., a blood sample, a PBMC sample, a synovial fluid sample or a synovium sample.

Set forth below are exemplary methods which can be used to determine the level of expression of one or more genes. In one embodiment for determining the level of expression of a plurality of genes, arrays, e.g., microarrays, can be used.

6.a. Use of Arrays for Determining the Level of Expression of Genes

Generally, determining expression profiles with arrays involves the following steps: (a) obtaining a mRNA sample from a subject and preparing labeled nucleic acids therefrom (the “target nucleic acids” or “targets”); (b) contacting the target nucleic acids with the array under conditions sufficient for target nucleic acids to bind with corresponding probes on the array, e.g. by hybridization or specific binding; (c) optionally removing unbound targets from the array; (d) detecting bound targets, and (e) analyzing the results.

(i) Obtaining a mRNA Sample of a Subject

In one embodiment, one or more cells from the subject to be tested are obtained and RNA is isolated from the cells. In one embodiment, PBMCs, synovial fluid, synovium or cartilage are obtained from the subject according to methods known in the art.

(ii) Labeling of the Nucleic Acids to be Analyzed

Generally, the target molecules will be labeled to permit detection of hybridization of target molecules to a microarray by methods known in the art.

In other embodiments, the target nucleic acid is not labeled. In this case, hybridization can be determined, e.g., by plasmon resonance In one embodiment, a plurality (e.g., 2, 3, 4, 5 or more) of sets of target nucleic acids are labeled and used in one hybridization reaction (“multiplex” analysis).

The use of a two-color fluorescence labeling and detection scheme to define alterations in gene expressionhas been described previously. An advantage of using cDNA labeled with two different fluorophores is that a direct and internally controlled comparison of the mRNA levels corresponding to each arrayed gene in two cell states can be made, and variations due to minor differences in experimental conditions (e.g, hybridization conditions) will not affect subsequent analyses.

The quality of labeled nucleic acids can be evaluated prior to hybridization to an array. For example, a sample of the labeled nucleic acids can be hybridized to probes derived from the 5′, middle and 3′ portions of genes known to be or suspected to be present in the nucleic acid sample. This will be indicative as to whether the labeled nucleic acids are full length nucleic acids or whether they are degraded. In one embodiment, the GeneChip® Test3 Array from Affymetrix (Santa Clara, Calif.) can be used for that purpose. This array contains probes representing a subset of characterized genes from several organisms including mammals. Thus, the quality of a labeled nucleic acid sample can be determined by hybridization of a fraction of the sample to an array, such as the GeneChip® Test3 Array from Affymetrix (Santa Clara, Calif.).

(iii) Exemplary arrays

Arrays, e.g., microarrays, for use according to the invention include one or more probes of genes which are up- or down-regulated in R.A. In one embodiment, the array comprises probes corresponding to one or more of genes selected from the group consisting of genes which are up-regulated in R.A., e.g., genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6 and genes which are down-regulated, e.g., CAMK2B, PLA2G2A, GBAS and SOX15. The array may comprise probes corresponding to at least 10, at least 20, at least 50, at least 100 or at least 1000 genes.

There can be one or more than one probe corresponding to each gene on a microarray. For example, a microarray may contain from 2 to 20 probes corresponding to one gene and in some embodiments about 5 to 10. The probes may correspond to the full length RNA sequence or complement thereof of genes characteristic of R.A., or they may correspond to a portion thereof, which portion is of sufficient length for permitting specific hybridization. Such probes may comprise from about 50 nucleotides to about 100, 200, 500, or 1000 nucleotides or more than 1000 nucleotides. As further described herein, microarrays may contain oligonucleotide probes, consisting of about 10 to 50 nucleotides, or about 15 to 30 nucleotides or about 20-25 nucleotides. The probes may be single stranded. The probe will have sufficient complementarity to its target to provide for the desired level of sequence specific hybridization (see below).

Typically, the arrays used in the present invention will have a site density of greater than 100 different probes per cm2. Microarrays can be prepared by methods known in the art, or they can be custom made by companies, e.g., Affymetrix (Santa Clara, Calif.).

(iv) Detection of Hybridization and Analysis of Results

The above steps result in the production of hybridization patterns of target nucleic acid on the array surface. These patterns may be visualized or detected in a variety of ways, with the particular manner of detection being chosen based on the particular label of the target nucleic acid. Representative detection means include scintillation counting, autoradiography, fluorescence measurement, colorimetric measurement, light emission measurement, light scattering, and the like.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution multi-color images, flagging or removing artifacts, verifying that controls have performed properly, normalizing the signals, interpreting fluorescence data to determine the amount of hybridized target, normalization of background and single base mismatch hybridizations, and the like. In one embodiment, a system comprises a search function that allows one to search for specific patterns, e.g., patterns relating to differential gene expression, e.g., between the expression profile of a cell of R.A. and the expression profile of a counterpart normal cell in a subject. A system allows one to search for patterns of gene expression between more than two samples.

A desirable system for analyzing data is a general and flexible system for the visualization, manipulation, and analysis of gene expression data. Such a system includes a graphical user interface for browsing and navigating through the expression data, allowing a user to selectively view and highlight the genes of interest. The system also may include sort and search functions and may be available for general users with PC, Mac or Unix workstations. Also included in the system may be clustering algorithms that are qualitatively more efficient than existing ones. The accuracy of such algorithms may be hierarchically adjustable so that the level of detail of clustering can be systematically refined as desired.

Various algorithms are available for analyzing the gene expression profile data, e.g., the type of comparisons to perform. In certain embodiments, it is desirable to group genes that are co-regulated. This allows the comparison of large numbers of profiles. One embodiment for identifying such groups of genes involves clustering algorithms. In some embodiments, a gene expression profile is converted to a projected gene expression profile. The projected gene expression profile is a collection of geneset expression values. The conversion is achieved, in some embodiments, by averaging the level of expression of the genes within each geneset. In some other embodiments, other linear projection processes may be used. The projection operation expresses the profile on a smaller and biologically more meaningful set of coordinates, reducing the effects of measurement errors by averaging them over each cellular constituent sets and aiding biological interpretation of the profile.

Values that can be compared include gross expression levels; averages of expression levels, e.g., from different experiments, different samples from the same subject or samples from different subjects; and ratios of expression levels, e.g., between R.A. subjects and normal controls, between different R.A. subjects and isolated cell populations.

6. b. Other Methods for Determining Gene Expression Levels

Other methods for determining gene expression levels have been described in Section 3.b.

6.c. Data Analysis Methods

Comparison of the expression levels of one or more genes which are up- or down-regulated in R.A. with reference expression levels, e.g., expression levels in cells characteristic of R.A. or in normal counterpart cells, may be conducted using computer systems. In one embodiment, one or more expression levels are obtained in two cells and these two sets of expression levels are introduced into a computer system for comparison. In one embodiment, one set of one or more expression levels is entered into a computer system for comparison with values that are already present in the computer system, or in computer-readable form that is then entered into the computer system.

6.d. Exemplary Diagnostic and Prognostic Compositions and Devices of the Invention

Any composition and device (e.g., an array) used in the above-described methods are within the scope of the invention.

In one embodiment, the invention provides a composition comprising a plurality of detection agents for detecting expression of genes which are up- or down-regulated in R.A. In one embodiment, the composition comprises at least 2, at least 3, 5, 10, 20, 50, or 100 different detection agents. A detection agent can be a nucleic acid probe, e.g., DNA or RNA, or it can be a polypeptide, e.g., as antibody that binds to the polypeptide encoded by a gene characteristic of R.A. The probes can be present in equal amount or in different amounts in the solution.

A nucleic acid probe can be at least about 10 nucleotides long, or at least about 15, 20, 25, 30, 50, 100 nucleotides or more, and can comprise the full length gene. If the nucleic acid is short (i.e., 20 nucleotides or less), the sequence may be perfectly complementary to the target gene (i.e., a gene that is characteristic of R.A.), such that specific hybridization can be obtained. However, nucleic acids, even short ones that are not perfectly complementary to the target gene can also be included in a composition of the invention, e.g., for use as a negative control. Certain compositions may also comprise nucleic acids that are complementary to, and capable of detecting, an allele of a gene.

In one embodiment, the invention provides nucleic acids which hybridize under high stringency conditions of 0.2 to 1×SSC at 65° C. followed by a wash at 0.2×SSC at 65° C. to genes which are up- or down-regulated in R.A. In another embodiment, the invention provides nucleic acids which hybridize under low stringency conditions of 6×SSC at room temperature followed by a wash at 2×SSC at room temperature. Other nucleic acids probes hybridize to their target in 3×SSC at 40 or 50° C., followed by a wash in 1 or 2×SSC at 20, 30, 40, 50, 60, or 65° C.

Nucleic acids which are at least about 80%, at least about 90%, at least about 95% orat least about 98% identical to genes which are up- or down-regulated in R.A. or cDNAs thereof, and complements thereof, are also within the scope of the invention.

Nucleic acid probes can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments. Computer programs can be used in the design of primers with the required specificity and optimal amplification properties. See, e.g., Oligo version 5.0 (National Biosciences).

In another embodiment, the invention provides a composition comprising a plurality of agents which can detect a polypeptide encoded by a gene characteristic of R.A. An agent can be, e.g., an antibody. Antibodies to polypeptides described herein can be obtained commercially, or they can be produced according to methods known in the art.

The probes can be attached to a solid support, such as paper, membranes, filters, chips, pins or glass slides, or any other appropriate substrate, such as those further described herein. For example, probes of genes which are up- or down-regulated in R.A. can be attached covalently or non covalently to membranes for use, e.g., in dotblots, or to solids such as to create arrays, e.g., microarrays.

### EXAMPLES

The present invention is further illustrated by the following examples which should not be construed as limiting in any way. The practice of the present invention will employ, unless otherwise indicated, conventional techniques of cell biology, cell culture, molecular biology, transgenic biology, microbiology, recombinant DNA, and immunology, which are within the skill of the art.

### Example 1

### Identification of Genes that are Up- or Down-Regulated in Patients Having Rheumatoid Arthritis

This Example describes the identification of several genes which are up- or downregulated in peripheral blood mononuclear cells (PBMCs) of subjects having rheumatoid arthritis (R.A.) relative to expression in PBMCs of normal subjects.

PMBCs were isolated form 9 patients with R.A. and 13 normal volunteers as follows. Eight mls of blood were drawn into a CPT Vacutainer tube which was inverted several times. The tube was centrifuged at 1500×g (2700 rpm) in a swinging bucket rotor at room temperature. The serum was removed and PBMCs were transferred to a 15 ml conical centrifuge tube. The cells were washed with the addition of phosphate buffered saline (PBS) and centrifuged at 450 g (1200 rpm) for 5 minutes. The supernatant was discarded and the wash procedure was repeated once more. After removal of the supernatant, total RNA was isolated with the use of the RNeasy minikit, (Qiagen, Hidden, Germany) according to the manufacturers procedure.

RNA was analyzed on oligonucleotide arrays composed of 6,800 and 12,000 human genes (Affymetrix Hu6800 and HgU95A chip sets, respectively), as follows.

Target nucleic acid for hybridization was prepared as follows. Total RNA was prepared for hybridization by denaturing 5 μg of total RNA from PBMC's for 10 minutes at 70° C. with 100 pM T7/T24-tagged oligo-dt primer (synthesized at Genetics Institute, Cambridge, Mass.), and cooled on ice. First strand cDNA synthesis was performed under the following buffer conditions: 1× first strand buffer (Invitrogen Life Technologies, Carlsbad, Calif.), 10 mM DTT(GIBCO/Invitrogen), 500 μM of each dNTP (Invitrogen Life Technologies), 400 units of Superscript RT II (Invitrogen Life Technologies) and 40 units RNAse inhibitor (Ambion, Austin, Tex.). The reaction proceeded at 47° C. for 1 hour. Second strand cDNA was synthesized with the addition of the following reagents at the final concentrations listed: 1× second strand buffer (Invitrogen Life Technologies), an additional 200 μM of each dNTP (Invitrogen Life Technologies), 40 units of E. coli DNA polymerase 1 (Invitrogen Life Technologies), 2 units E. coli RNaseH (Invitrogen Life Technologies), and 10 units of E. coli DNA ligase. The reaction proceeded at 15.8° C. for 2 hours and during the last five minutes of this reaction 6 units of T4 DNA polymerase (New England Biolabs, Beverly, Mass.) was added. The resulting double stranded cDNA was purified with the use of BioMag carboxyl terminated particles as follows: 0.2 mg of BioMag particles (Polysciences Inc., Warrington, Pa.) were equilibrated by washing three times with 0.5M EDTA and resuspended at a concentration of 22.2 mg/ml in 0.5M EDTA. The double stranded cDNA reaction was diluted to a final concentration of 10% PEG/1.25M NaCl and the bead suspension was added to a final bead concentration of 0.614 mg/ml. The reaction was incubated at room temperature for 10 minutes. The cDNA/bead complexes were washed with 30011 of 70% ethanol, the ethanol was removed and the tubes were allowed to air dry. The cDNA was eluted with the addition of 20 μl of 10 mM Tris-acetate, pH 7.8, incubated for 2-5 minutes and the cDNA containing supernatant was removed.

10 μl of purified double stranded cDNA was then added to an in vitro transcription (IVT) solution which contained, 1×IVT buffer (Ambion, Austin, Tex.) 5,000 units T7 RNA polymerase (Epicentre Technologies, Madison, Wis.), 3 mM GTP, 1.5 mM ATP, 1.2 mM CTP and 1.2 mM UTP (Amersham/Phammacia,), 0.4 mM each bio-16 UTP and bio-11 CTP (Enzo Diagnostics, Farmingdale, N.Y.), and 80 units RNase inhibitor (Ambion, Austin, Tex.). The reaction proceeded at 37° C. for 16 hours. Labeled RNA was purified with the use of an RNeasy (Qiagen). The RNA yield was quantitated by measuring absorbance at 260 nm.

Array Hybridization and Detection of Fluorescence was performed as follows. 12 μg of IVT was fragmented in 40 mM Tris-acetate, pH 8.0, 100 mM potassium acetate, and 30 mM magnesium acetate for 35 minutes at 94° C. The fragmented, labeled RNA probes were diluted in hybridization buffer at a final composition of 1×2-N-Morpholinoethanesulfonic acid (MES (buffer (pH 6.5), 50 pM Bio948 (control biotinylated oligo that hybridizes to landmark features on the probe array (Genetics Institute, Cambridge, Mass.)), 100 μg/ml herring sperm DNA (Promega, Madison, Wis.), 500 μg/ml acetylated BSA (Invitrogen Life Technologies) and 1 μl/μg standard curve reagent (Proprietary reagent supplied by Gene Logic, Gaithersburg, Md.). This hybridization solution was pre-hybridized with two glass beads (Fisher Scientific, Pittsburgh, Pa.) at 45° C. overnight. The hybridization solution was removed to a clean tube, heated for 1-2 min at 95° C. and microcentrifuged on high for 2 minutes to pellet insoluble debris. Affymetrix oligonucleotide array cartridges (human 6800 array P/N900183 and human U95A (Affymetrix, Santa Clara, Calif.)) were pre-wet with non-stringent wash buffer (0.9M NaCl, 60 mM sodium phosphate, 6 mM EDTA and 0.01% Tween20) and incubated at 45° C. with rotation for 5-10 minutes. Buffer was removed from the Affymetrix cartridges and the arrays were hybridized with 180 μl of the hybridization solution at 45° C. rotating at 45-60 rpm overnight. After overnight incubation, the hybridization solutions were removed and the cartridges were filled with non-stringent wash buffer. The array cartridges were washed using an Affymetrix fluidics station according with 10 cycles of 2 mixes/cycle non-stringent wash buffer at 25° C. followed by 4 cycles of 15 mixes/cycle stringent wash buffer (100 mM MES, 0.1M Na⁺, 0.01% Tween20 and 0.005% antifoam). The probe array was then first stained for 10 minutes at 25° C. in SAPE solution (100 mM MES, 1M Na⁺, 0.05% Tween20, 0.005% antifoam, 2 mg/ml acetylated BSA (Invitrogen Life Technologies) and 10 μg/ml R phycoerythrin streptavidin (Molecular Probes, Eugene, Oreg.)). After first staining, the probe array was washed for 10 cycles of 4 mixes/cycle with non-stringent wash buffer at 25° C. The probe array was then stained for 10 minutes at 25° C. in antibody solution (100 mM MES, 1M Na⁺, 0.05% Tween20, 0.005% antifoam, 2 mg/ml acetylated BSA (Invitrogen Life Technologies), 1001 g/ml Goat IgG (SIGMA, St. Louis, Mo.) and 3 μg/ml biotinylated anti-streptavidin antibody (goat) (Vector Laboratories). Following the second stain, the probe array is stained again for an additional 10 minutes at 25° C. in SAPE solution. Finally, the probe array is washed for 15 cycles of 4 mixes/cycle with non-stringent wash buffer at 30° C. Arrays were scanned using an Affymetrix gene chip scanner (Affymetrix, Santa Clara, Calif.). The scanner contains a scanning confocal microscope and uses an argon ion laser for the excitation source and emission is detected by a photomultiplier tube at 530 nm bandpass filter (fluorescein 0 or 560 longpass filter (phycoerythrin).

Data analysis was performed using GENECHIP 3.0 or 4.0 software with normalizing/scaling to internal controls. For each patient, two parameters were used to filter the data: 1) “Absolute Decision,” which indicates the presence (P) or absence (A) of RNA of a gene within a given RNA sample; 2) “Frequency,” which measures the number of copies of a given RNA within a RNA sample, and this value is expressed as Copies per million transcripts. If a gene was called “Absent,” its frequency was not used to calculate the average frequency of the gene. If a gene was called “Absent” for more than four patients in the Hu6800 data; more than two patients in the HgU95A data, or more than six normals, no average frequency was calculated. Genes that had average frequencies for normal volunteers only were tagged “Normal” while those that had average frequencies for patients only were tagged “Disease.” The fold change in gene expression was calculated by dividing the average gene frequency of the patients by that of the normals. Genes selected for analysis met the following criteria: 1) a fold change greater than 1.95 or less than −1.95 and 2) those genes tagged as either “Normal” or “Disease.”

### Example 2

### Identification of Genes which are Up- or Down-Regulated in an Animal Model of Rheumatoid Arthritis

This example describes the identification of several genes which are up- or down-regulated in mice having collagen induced arthritis (CIA) relative to normal mice. Gene expression was measured in paws of mice; PBMCs and in synovium.

CIA is an accepted animal model for rheumatoid arthritis. The disease was induced as follows in mice. Male DBA/1 (Jackson Laboratories, Bar Harbor, Me.) mice were used for all experiments. Arthritis was induced with the use of either chicken collagen type II (Sigma, St. Louis, Mo.) or bovine collagen type II (Chondrex, Redmond, Wash.). Chicken collagen was dissolved in 0.01 M acetic acid and emulsified with an equal volume of Complete Freund's adjuvant (CFA; Difco Labs, Detroit, Mich.) containing 1 mg/ml Mycobacterium tuberculosis (strain H37RA). 200 μg of chicken collagen was intradermally injected in the base of the tail on day 0. On day 21, mice were injected intraperitoneally with a PBS solution containing 100 μg of chicken collagen II. Bovine collagen type II (Chondrex, Redmond, Wash.) was dissolved in 0.1 M acetic acid and emulsified in an equal volume of CFA (Sigma) containing 1 mg/ml Mycobacterium tuberculosis (strain H37RA). 200 μg of bovine collagen was injected subcutaneously in the base of the tail on day 0. On day 21, mice were injected subcutaneously, in the base of the tail, with a solution containing 200 μg of bovine collagen in 0.1 M acetic acid that had been mixed with an equal volume of Incomplete Freund's adjuvant (Sigma). Naïve animals received the same sets of injections, minus collagen. Mice were monitored at least three times a week for disease progression. Individual limbs were assigned a clinical score based on the index: 0=normal; P=prearthritic, characterized by focal erythema on the tips of digits.; 1=visible erythema accompanied by 1-2 swollen digits.; 2=pronounced erythema, characterized by paw swelling and/or multi digit swelling.; 3=massive swelling extending into ankle or wrist joint.; 4=difficulty in use of limb or joint rigidity. The sum of all limb scores for any given mouse could yield a maximum total body score of 16.

At various stages of disease, animals were euthanized and tissues were harvested. In one series of examples, at least two paws from each animal were flash frozen in liquid nitrogen for RNA analyses. Frozen mouse paws were pulverized to a fine powder with the use of a mortar and pestle and liquid nitrogen. RNA was purified using the Promega RNAgents Total RNA Isolation System (Promega, Madison, Wis.). The RNA w further purified using the RNeasy minikit. The remaining paws were fixed in 10% formalin for histology.

In another series of examples, gene expression was determined in PBMCs of mice. Blood was collected via cardiac puncture into EDTA coated collection tubes. Blood samples were pooled according to similar total body scores (normal, prearthritic, scores 1, 3, 4, 5, 6, and 7-9) into a 15 ml conical tube. The blood was diluted 1:1 with PBS that contained 2 mM EDTA, and layered on an equal volume of Lympholyte-M (Cedar Lane Labs, Homby, Ontario, Canada). The mixture was centrifuged, with no brake, for 20 minutes at 1850 rpm in a Sorvall centrifuge, (model RT 6000D). Cells at the interface were collected and added to a new tube. The cells were washed with the addition of 10 ml PBS, containing 2 mM EDTA, and centrifuged at 1200 rpm for 10 minutes. The wash was repeated two times. To lyse residual red cells, cell pellets were dispersed in 2 ml of cold 0.2% NaCl and incubated on ice for 45-60 seconds. Lysis was terminated with the addition of 2 ml of 1.6% NaCl and the cells were centrifuged at 1200 rpm for 10 minutes. PBMCs were resuspended in 5 ml of PBS, which contained 2 mM EDTA, and counted. Cells were centrifuged at 1200 rpm for 10 minutes, and the supernatant discarded in preparation for RNA isolation. Total RNA was isolated from the PBMCs using the RNeasy minikit (Qiagen, Hidden, Germany).

In yet another series of examples, RNA was obtained from isolated synovium of the diseased animals. The joint synovium was dissected from diseased and control animals under a dissection scope. Tissues from five or more animals with similar disease scores were pooled and RNA was isolated using the RNeasy kit (Qiagen, Hidden, Germany).

Gene expression was analyzed on the oligonucleotide arrays Affymetrix murine 11K chip set composed of 11,000 murine genes on two chips, murine 11KsubA P/N 900188 and murine 11 KsubB P/N900189.

Labeled target nucleic acids for hybridization to the chips were prepared as described in the previous Example with 5 μg of PBMC RNA or 7 μg of RNA from paws or synovial tissue.

Data analysis was performed using GENECHIP 3.0 software with normalizing/scaling to internal controls. Each experimental sample was compared to a time matched control in a two-file analysis. Next, the data were entered into the GeneSpring (Silicon Genetics, Redwood City, Calif.) analysis program. The data were filtered in a hierarchical fashion. First, the data were grouped according to paw scores. For each score, a list of genes that were called “Present” in all samples in a given score group and in the control was created. These lists were further refined by removing all genes that were not called either “Increasing” or “Decreasing” (defined in the program) in at least a majority of the samples in each score group. These lists were then filtered for genes that showed fold change greater than or equal to 1.95 or less than or equal to −1.95 in either all of the samples, if there were less than five samples, or in greater than 70% of the samples.

The results show that several genes, e.g., PTPN18, HMG-1 and SLPI, that are significantly up-regulated in the mouse model, were also significantly up-regulated in human PBMCs of R.A. patients.

### Example 3

### Identification of Cells Expressing Genes which are Up-Regulated in R.A.

This Example describes the identity of cells expressing genes which are up-regulated in R.A. by in situ hybridization.

Paws of CIA mice were fixed in 4% paraformaldeyde, pH 7.47, decalcified in 20% EDTA (pH 8.0) and embedded in paraffin for in situ hybridization according to methods known in the art.

Sense and anti-sense riboprobes for use in the in situ hybridization were produced by generating 2 independent PCR products, as follows. T7 RNA polymerase binding sites were incorporated into the oligonucleotides to insert T7 binding sites at either the 5′end of the PCR product for sense riboprobe or the 3′ end of the PCR product for antisense riboprobe. Digoxygenin labeled probes were prepared with the use of a DIG RNA labeling mix (Roche Diagnostics, Mannheim, Germany), as described by the manufacturer, and T7 RNA polymerase (Roche Diagnostics).

The probes were obtained by PCR using the following oligonucleotide primers for each of the sense and antisense probe.

Murine SAA3 Sense Riboprobe:

Forward Primer (with T7 Site):

| | |
|---|---|
| 5′GACTGATAATACGACTCACTATAGGGCGAATGAA | (SEQ ID NO: 1) |
| GCCTTCCATTGCCATCATTCTTTGCA3′ | |

Reverse Primer:

(SEQ ID NO: 2)

5′TTAGCGGCCGCTCAGTATCTTTTAGGCAGGCCAGCAGGTCGGAA3′

The probe sequence covers the entire coding sequence, is 369 nucleotides long and has the following sequence:

| | |
|---|---|
| atgaagcctt ccattgccat cattctttgc atcttgatcc tgggagttga cagccaaaga tgggtccagt tcatgaaaga | (SEQ ID NO: 3) |
| agctggtcaa gggtctagag acatgtggcg agcctactct gacatgaaga aagctaactg gaaaaactca gacaaatact | |
| tccatgctcg ggggaactat gatgctgccc ggaggggtcc cgggggagcc tgggctgcta aagtcatcag cgatgccaga | |
| gaggctgttc agaagttcac gggacatgga gcagaggact caagagctga ccagtttgcc aatgagtggg gccggagtgg | |
| caaagacccc aaccacttcc gacctgctgg cctgcctaaa agatactga | |

Murine SAA3 Anti-Sense Riboprobe:

Forward Primer:

(SEQ ID NO: 4)

5′TTAGAATTCATGAAGCCTTCCATTGCCATCATTCTTTGCA3′

Reverse Primer (with T7 Site):

| | |
|---|---|
| 5′GACTGATAATACGACTCACTATAGGGCGATCAGT | (SEQ ID NO: 5) |
| ATCTTTTAGGCAGGCCAGCAGGTCGGAA3′ | |

The probe sequence covers the entire coding sequence, is 369 nucleotides long and has the following sequence:

| | |
|---|---|
| tcagtatctt ttaggcaggc cagcaggtcg gaagtggttg gggtctttgc cactccggcc ccactcattg gcaaactggt | (SEQ ID NO: 6) |
| cagctcttga gtcctctgct ccatgtcccg tgaacttctg aacagcctct ctggcatcgc tgatgacttt agcagcccag | |
| gctcccccgg gacccctccg ggcagcatca tagttccccc gagcatggaa gtatttgtct gagtttttcc agttagcttt | |
| cttcatgtca gagtaggctc gccacatgtc tctagaccct tgaccagctt ctttcatgaa ctggacccat ctttggctgt | |
| caactcccag gatcaagatg caaagaatga tggcaatgga aggcttcat | |

Murine LST-1 Sense Riboprobe:

Forward primer (with T7 Site):

| | |
|---|---|
| 5′GACTGATAATACGACTCACTATAGGGCGAATGTC | (SEQ ID NO: 7) |
| TGATGACAATGGATCTGGTAACAATTGCA3′ | |

Reverse Primer:

(SEQ ID NO: 8)

5′TTAGCGGCCGCTCAAGTGGGTGTGCTCCTGGCGATGCAGGCATA3′

The probe sequence covers the entire coding sequence, has 288 nucleotides and the following sequence:

| | |
|---|---|
| atgtctgatg acaatggatc tggtaacaat tgcacaacca atcatttcct gctctatggg agcctgggac tgggagggct | (SEQ ID NO: 9) |
| cctcctcctg cttgtcatca tcctgttcat ctgcctgtgc gggttcagtc agagagtgaa gagactggaa aggaatgccc | |
| aggtctcagg gcaggagccc cactatgcat ctctccagca gctgccagtg tccagtagtg atatcacaga catgaaagaa | |
| gacctcagca ctgactatgc ctgcatcgcc aggagcacac ccacttga | |

Murine LST-1 Anti-Sense Riboprobe:

Forward Primer:

(SEQ ID NO: 10)

5′TTAGAATTCATGTCTGATGACAATGGATCTGGTAACAATTGCA3′

Reverse Primer (with T7 Site):

| | |
|---|---|
| 5′GACTGATAATACGACTCACTATAGGGCGATCA | (SEQ ID NO: 11) |
| AGTGGGTGTGCTCCTGGCGATGCAGGCATA3′ | |

The probe sequence covers the entire coding sequence, is 288 nucleotides long and has the following sequence:

| | |
|---|---|
| tcaagtgggt gtgctcctgg cgatgcaggc atagtcagtg ctgaggtctt ctttcatgtc tgtgatatca | (SEQ ID NO: 12) |
| ctactggaca ctggcagctg ctggagagat gcatagtggg gctcctgccc tgagacctgg gcattccttt | |
| ccagtctctt cactctctga ctgaacccgc acaggcagat gaacaggatg atgacaagca ggaggaggag | |
| ccctcccagt cccaggctcc catagagcag gaaatgattg gttgtgcaat tgttaccaga tccattgtca | |
| tcagacat | |

Murine FSTI Sense Riboprobe:

Forward Primer (with T7 Site):

| | |
|---|---|
| 5′GACTGATAATACGACTCACTATAGGGCGAATG | (SEQ ID NO: 13) |
| TGGAAACGATGGCTGGCGCTCTCGCTG3′ | |

Reverse Primer:

| | |
|---|---|
| 5′AGGAACAGACACAGCGATTGC3′ | (SEQ ID NO: 14) |

The probe is 421 nucleotides long and has the following sequence:

| | |
|---|---|
| atgtggaaac gatggctggc gctctcgctg gtgaccatcg ccctggtcca cggcgaggag gaacctagaa | (SEQ ID NO: 15) |
| gcaaatccaa gatctgcgcc aatgtgtttt gtggagctgg cagggaatgt gccgtcacag agaaggggga | |
| gcccacgtgc ctctgcattg agcaatgcaa acctcacaag aggcctgtgt gtggcagtaa tggcaagacc | |
| tacctcaacc actgtgaact tcatagagat gcctgcctca ctggatccaa gatccaggtt gattatgatg | |
| ggcactgcaa agaaaagaag tctgcgagtc catctgccag cccagttgtc tgctatcaag ctaaccgcga | |
| tgagctccga cggcgcctca tccagtggct ggaagctgag atcattccag atggctggtt ctctaaaggc | |
| a | |

Murine FST1 Anti-Sense Riboprobe:

Forward Primer:

| | |
|---|---|
| 5′GGGGATATCATGTGGAAACGATGGCTGGCGCT | (SEQ ID NO: 16) |
| CTCGCTGGTGACCAT3′ | |

Reverse Primer (with T7 Site):

| | |
|---|---|
| 5′GACTGATAATACGACTCACTATAGGGCGATGC | (SEQ ID NO: 17) |
| CTTTAGAGAACCAGCCATCTGGAATGA3′ | |

The probe is 421 nucleotides long and has the following sequence:

| | |
|---|---|
| tgcctttaga gaaccagcca tctggaatga tctcagcttc cagccactgg atgaggcgcc gtcggagctc | (SEQ ID NO: 18) |
| atcgcggtta gcttgatagc agacaactgg gctggcagat ggactcgcag acttcttttc tttgcagtgc | |
| ccatcataat caacctggat cttggatcca gtgaggcagg catctctatg aagttcacag tggttgaggt | |
| aggtcttgcc attactgcca cacacaggcc tcttgtgagg tttgcattgc tcaatgcaga ggcacgtggg | |
| ctcccccttc tctgtgacgg cacattccct gccagctcca caaaacacat tggcgcagat cttggatttg | |
| cttctaggtt cctcctcgcc gtggaccagg gcgatggtca ccagcgagag cgccagccat cgtttccaca | |
| t | |

Murine SLPI Sense Riboprobe:

Forward Primer (with T7 Site):

| | |
|---|---|
| 5′GACTGATAATACGACTCACTATAGGGCGAATG | (SEQ ID NO: 18) |
| AAGTCCTGCGGCCTTTTACCTTTCACGGTG3′ | |

Reverse Primer:

(SEQ ID NO: 19)

5′AATGCGGCCGCTCACATCGGGGGCAGGCAGACTTTCCCAC3′

The probe sequence covers the entire coding sequences, is 396 nucleotides long and has the following sequence:

| | |
|---|---|
| atgaagtcct gcggcctttt acctttcacg gtgctccttg ctctggggat cctggcaccc tggactgtgg | (SEQ ID NO: 20) |
| aaggaggcaa aaatgatgct atcaaaatcg gagcctgccc tgctaaaaag cctgcccagt gccttaagct | |
| tgagaagcca caatgccgta ctgactggga gtgcccggga aagcagaggt gctgccaaga tgcttgcggt | |
| tccaagtgcg tgaatcctgt tcccattcgc aaaccagtgt ggaggaagcc tgggaggtgc gtcaaaactc | |
| aggcaagatg tatgatgctt aaccctccca atgtctgcca gagggacggg cagtgtgacg gcaaatacaa | |
| gtgctgtgag ggtatatgtg ggaaagtctg cctgcccccg atgtga | |

Murine SLPI Anti-Sense Riboprobe:

Forward Primer:

(SEQ ID NO: 21)

5′ATTGAATTCATGAAGTCCTGCGGCCTTTTACCTTTCACGGTGC3′

Reverse Primer (with T7 Site):

| | |
|---|---|
| 5′GACTGATAATACGACTCACTATAGGGCGATCA | (SEQ ID NO: 22) |
| CATCGGGGGCAGGCAGACTTTCCCAC3′ | |

The probe sequence covers the entire coding equence, is 396 nucleotides long and has the following sequence:

| | |
|---|---|
| tcacatcggg ggcaggcaga ctttcccaca tataccctca cagcacttgt atttgccgtc acactgcccg | (SEQ ID NO: 23) |
| tccctctggc agacattggg agggttaagc atcatacatc ttgcctgagt tttgacgcac ctcccaggct | |
| tcctccacac tggtttgcga atgggaacag gattcacgca cttggaaccg caagcatctt ggcagcacct | |
| ctgctttccc gggcactccc agtcagtacg gcattgtggc ttctcaagct taaggcactg ggcaggcttt | |
| ttagcagggc aggctccgat tttgatagca tcatttttgc ctccttccac agtccagggt gccaggatcc | |
| ccagagcaag gagcaccgtg aaaggtaaaa ggccgcagga cttcat | |

Murine Legumain Sense Riboprobe:

Forward Primer (with T7 Site):

| | |
|---|---|
| 5′GACTGATAATACGACTCACTATAGGGCGA AC | (SEQ ID NO: 24) |
| ACCAACACCAGCCATGTC3′ | |

Reverse Primer:

| | |
|---|---|
| 5′CTCTCAGCAGTTTCCCCAAATC3′ | (SEQ ID NO: 25) |

The probe is 313 nucleotides long and has the following sequence:

| | |
|---|---|
| acaccaacac cagccatgtc atgcaatatg ggaacaaatc tatctctacc atgaaagtga tgcagtttca | (SEQ ID NO: 26) |
| gggaatgaag cacagagcca gttcccccat ctccctgcct ccggtcacac accttgacct cacccccagc | |
| cctgacgtgc ccctgaccat cttgaagagg aagctgctga gaaccaacga cgtgaaggaa tcccagaatc | |
| tcattgggca gatccagcaa tttctggatg ccaggcacgt cattgagaag tctgtgcaca agatcgtttc | |
| cctgctggcg ggatttgggg aaactgctga gag | |

Murine Legumain Anti-Sense Riboprobe:

Forward Primer:

| | |
|---|---|
| 5′ACACCAACACCAGCCATGTC3′ | (SEQ ID NO: 27) |

Reverse Primer (with T7 Site):

| | |
|---|---|
| 5′GACTGATAATACGACTCACTATAGGGCGACTC | (SEQ ID NO: 28) |
| TCAGCAGTTTCCCCAAATC3′ | |

The probe sequence is 313 nucleotides long and has the following sequence:

| | |
|---|---|
| ctctcagcag tttccccaaa tcccgccagc agggaaacga tcttgtgcac agacttctca atgacgtgcc | (SEQ ID NO: 29) |
| tggcatccag aaattgctgg atctgcccaa tgagattctg ggattccttc acgtcgttgg ttctcagcag | |
| cttcctcttc aagatggtca ggggcacgtc agggctgggg gtgaggtcaa ggtgtgtgac cggaggcagg | |
| gagatggggg aactggctct gtgcttcatt ccctgaaact gcatcacttt catggtagag atagatttgt | |
| tcccatattg catgacatgg ctggtgttgg tgt | |

Sections of paraffin embedded tissue were de-paraffinized with xylene, 2 changes, 3 minutes each, and rehydrated to water. After a rinse in RNase-free water and phosphate buffered saline (PBS), permeabilization was performed by incubation with 0.2% Triton-X 100/PBS for 15 minutes. After 2 washes with PBS, each at 3 minutes, the sections were ready for proteinase K (PK)(Sigma) treatment. Sections were immersed in 0.1M Tris and 50 mM EDTA (Sigma) (pH 8.0) pre-warmed at 37° C. containing 5 μg/ml PK for 15 minutes. PK activities were stopped by 0.1M glycine/PBS for 5 minutes followed by a post fixation with 4% paraformaldehyde for 3 minutes and PBS rinsed. To prevent non-specific electrostatic binding of the probe, sections were immersed in 0.25% acetic anhydride and 0.1M triethanolamine solution (pH 8.0) for 10 minutes, followed by 15 seconds in 20% acetic acid at 4° C. After 3 changes in PBS, 5 minutes each, sections were dehydrated through 70%, 90% and 100% ethanol, each at 3 minutes. The sections were completely air dried before 40 μl of pre-hybridization buffer was applied, covered with Parafilm and incubated at 52° C. for 30 minutes to reduce non-specific binding. Parafilm was removed and 40 μl of hybridization buffer containing 5 ng/μl of digoxigenin-labeled probe was applied to each section, recovered with Parafilm and incubated overnight at 52° C.

Parafilm was carefully removed and sections were immersed into 2× saline sodium citrate (SSC) (Sigma)/0.1% lauryl sulphate (SDS) (Sigma) at room temperature, 4 changes, 5 minutes each. To ensure specific binding of the probe, sections were washed in a high stringency solution containing 0.1×SSC/0.1% SDS at 52° C., 2 changes, 10 minutes each. Endogenous peroxidase was quenched by immersion of sections in 3% H₂O₂, 15 minutes at room temperature followed by 3 washes in PBS, 2 minutes each. The labeled probe was detected with anti-digoxigenin antibody conjugated to peroxidase complex (Roche) diluted 1:50 in 2% normal sheep serum/0.1% Triton X-100. Labeled probe was developed with DAB (Vector Laboratories), washed in water, stained briefly with hematoxylin, dehydrate in graded alcohol and mounted in Permount mountant (Fisher Scientific) before microscopic examination.

The results indicate that no staining was observed in any of the paws treated with the sense probes (negative control). Expression of all of the genes described below was detected in joints of animals with collagen induced arthritis. No staining was seen in untreated animals.

More particularly, individual cells expressing the RNAs tested for were identified. FST1 mRNA positive cells were neutrophils, macrophages, fibroblasts, osteoblasts. No FST1 was found in bone tissue. SAA-3 mRNA positive cells were, neutrophils, macrophages, fibroblasts, superficial epidermis and chondrocytes. No staining with SAA-3 was seen in the articular cartilage. SLP-1 mRNA positive cells were osteoblasts, fibroblast and a focal area of chondrogenesis. The macrophages seemed to be positive (mild), endothelial cells appeared to be positive (mild) and neutrophils seemed to be negative for SLP-1 mRNA. Lymphocytes were difficult to identify in the SLP-1 hybridized sections. Legumain mRNA positive cells were seen in the epidermis. Osteoblasts and fibroblast had positive cytoplasmic staining with the Legumain antisense probe. The macrophages were positive (mild) and neutrophils appeared to be negative for Legumain mRNA. Lymphocytes were difficult to identify in the Legumain hybridized sections.

Equivalents

The present invention provides in part methods of screening certain genes and their encoded gene products to develop modulators for treating rheumatoid arthritis. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entireties as if each individual publication or patent was specifically and individually indicated to be incorporated by reference. In case of conflict, the present application, including any definitions herein, will control.

1. A method for identifying or validating a candidate compound for treating or preventing rheumatoid arthritis comprising contacting the compound with a RAGE (AGER) gene or protein.

2. The method of claim 1, wherein the compound is selected from the group consisting of: proteins, peptides, peptidomimetics, nucleic acids and small molecules.

3. The method of claim 1, wherein the compound is in a library of compounds.

4. The method of claim 1, wherein binding is determined using an in vitro assay.

5. The method of claim 1, wherein binding is determined using an in vivo assay.

6. A method for identifying or validating a candidate compound for treating or preventing rheumatoid arthritis, comprising comparing the RAGE (AGER) expression profile of a cell incubated with a test compound with the RAGE (AGER) expression profile of a counterpart cell derived from a normal subject, wherein a similar expression profile in the two cells indicates that the compound is likely to be effective as a therapeutic for rheumatoid arthritis.

7. The method of claim 6, wherein the compound is selected from the group consisting of:
proteins, peptides, peptidomimetics, nucleic acids and small molecules.

8. The method of claim 6, wherein the compound is in a library of compounds.

9. The method of claim 6, wherein binding is determined using an in vitro assay.

10. The method of claim 6, wherein binding is determined using an in vivo assay.
